(19) United States
(12) Patent Application Publication
(10) Pub. No.: US 2025/0191716 A1
(43) Pub. Date: Jun. 12, 2025

(54) SYNTHESIZER COMPUTING MATRICES FOR PROTOCOL GENERATION

(71) Applicant: Plesio Health LLC, Chicago, IL (US)

(72) Inventor: Rebecca Abraham, Skokie, IL (US)

(21) Appl. No.: 18/972,561

(22) Filed: Dec. 6, 2024

Related U.S. Application Data
(60) Provisional application No. 63/607,086, filed on Dec. 6, 2023.

(51) Int. Cl. G16H 20/10; G06F 5/01; G16C 20/10; G16H 15/00

(52) U.S. Cl. CPC G16H 20/10; G06F 5/01; G16C 20/10; G16H 15/00

(57) ABSTRACT

The disclosed methods include: determining or initializing an anchor computing model, an additive computing model, and a biometric computing model which are communicatively coupled to create a synthesizer computing matrix; receiving a computing input; and generating a synthesis protocol based on the synthesizer computing matrix and the computing input, wherein the synthesis protocol is comprised in a generated file or document including one or more of: a first dosing procedure of a first composition associated with the first digital profile; a first unidimensional or multidimensional digital representation of the first composition; a first dosage amount of the first composition; a first method of delivery of the first composition; first response data associated with the first method of delivery of the first composition; and first safety data indicating precautionary measures before, during, or after applying the first method of delivery of the first composition.

### CROSS-REFERENCE TO RELATED APPLICATIONS

This application claims priority to U.S. Provisional Application No. 63/607,086, filed on Dec. 6, 2023, titled “Dynamic Methods For Analyzing And Communicating Cannabinoid Data In A Data Network,” which is incorporated herein by reference in its entirety for all purposes.

### TECHNICAL FIELD

The present disclosure relates to methods, systems, and computer programs for optimizing composition data generation and analysis in association with titration and delivery operations for chemical compositions.

### BACKGROUND

Botanicals like cannabis and state-regulated medical marijuana and recreational marijuana with Tetrahydrocannabinol (THC), hemp-derived compositions, hemp-extracted compositions, and other plant-derived supplements and medicines behave differently than a majority of pharmaceutical compounds and/or supplements when applied to: different classes of biological systems; and/or different biological system demographics; and/or different biological systems with different health conditions.

There is a need for optimal methods and systems, and/or uniform methods and systems for quantitative and/or qualitative metric generation that accurately characterize or otherwise determine cannabinoid dosages (e.g., dosage amounts) and/or processes for administering said dosages to users/biological systems belonging to specific user classes or demographics. In particular, there is a need for tools and/or methodologies that facilitate effectively gauging, titrating, or otherwise customizing cannabinoid or THC dosages for specific biological systems or user classes.

Furthermore, there is a need for a dosing tool and/or cannabinoid assessment tool that not only drives research (e.g., composition research, consumer research, etc.) but also provides cannabinoid usage data insights including user safety and usage risk consideration data associated with titrating and/or administering cannabis or other THC compositions.

### SUMMARY

Disclosed are methods, systems, and computer program products for generating a synthesis protocol associated with a digital profile. According to an embodiment, a method for generating a synthesis protocol associated with a digital profile comprises: determining or initializing an anchor computing model configured to indicate control data associated with at least a first digital profile; determining or initializing an additive computing model configured to indicate effects data associated with the at least the first digital profile; and determining or initializing, a biometric computing model configured to indicate sensor measurements associated with the first digital profile.

The method further comprises: communicatively coupling the anchor computing model, the additive computing model, and the biometric computing model and thereby creating a synthesizer computing matrix; determining, based on the synthesizer computing matrix, a first qualitative parameter set associated with the anchor computing model along a first dimension; determining, based on the synthesizer computing matrix, a second qualitative parameter set associated with the additive computing model along the first dimension; determining, based on the synthesizer computing matrix, a third qualitative parameter set associated with the biometric computing model along the first dimension; and mapping, based on the synthesizer computing matrix, one or more qualitative data categories across: the anchor computing model in a second dimension; the additive computing model in the second dimension; and the biometric computing model in the second dimension.

According to one embodiment, the method comprises: receiving, based on the synthesizer computing matrix, a first computing input; normalizing, based on the synthesizer computing matrix, the first computing input thereby generating: a first normalized data value indicating the control data associated with the first digital profile, a second normalized data value indicating the effects data associated with the first digital profile, and a third normalized data value indicating the sensor measurements associated with the first digital profile.

The method also comprises selectively transforming, based on the synthesizer computing matrix: the first normalized data value into a first numerical data value or a first numerical data range or a first numerical data relation corresponding to a first aligned qualitative data category comprised in the one or more qualitative data categories; the second normalized data value into a second numerical data value or a second numerical data range or a second numerical data relation corresponding to a second aligned qualitative data category comprised in the one or more qualitative data categories; and the third normalized data value into a third numerical data value or a third numerical data range or a third numerical data relation corresponding to a third aligned qualitative data category comprised in the one or more qualitative data categories.

In one embodiment, the method comprises projecting, based on the synthesizer computing matrix: one or more of the first numerical data value or the first numerical data range or the first numerical data relation thereby generating an anchor parameter; one or more of the second numerical data value or the second numerical data range or the second numerical data relation thereby generating an additive parameter; and one or more of the third numerical data value or the third numerical data range or the third numerical data relation thereby generating a biometric parameter.

According to some embodiments, the method comprises synthesizing or combining, based on the synthesizer computing matrix, the anchor parameter, the additive parameter, and the biometric parameter, thereby generating a first synthesis protocol associated with the first digital profile, the synthesis protocol being comprised in a first file or document including at least three of: a first dosing procedure of a first composition associated with the first digital profile; a first unidimensional or multidimensional digital representation of the first composition; a first dosage amount of the first composition; a first method of delivery of the first composition; first response data associated with the first method of delivery of the first composition; and first safety data indicating precautionary measures before, during, or after applying the first method of delivery of the first composition.

Additionally, the method comprises transmitting the first file or document within which is comprised the synthesis protocol, to at least one of: a first computing device configured to visualize or display the synthesis protocol; a first dosing control system configured to implement the first dosing procedure and thereby generate the first composition; a first delivery control system configured to implement or initiate the first method of delivery of the first composition into a biological system associated with the first digital profile.

In other embodiments, a system and a computer program can include or execute the method described above. These and other implementations may each optionally include one or more of the following features.

The first qualitative parameter set associated with the anchor computing model comprises one or more of: a symptom control property configured to characterize biological symptoms from known or unknown causes associated with the first digital profile; a psychosomatic property configured to characterize psychological or emotional data associated with the first digital profile; and a somatic property configured to characterize physiological data associated with the first digital profile.

It is appreciated that the second qualitative parameter set associated with the additive computing model comprises one or more of: euphoria response data associated with the first composition or a second composition relative to the first digital profile; drowsiness data associated with the first composition or the second composition relative to the first digital profile; and appetite stimulation data associated with the first composition or the second composition relative to the first digital profile.

In some embodiments, the third qualitative parameter set associated with the biometric computing model comprises one or more of: heart rate data associated with the first digital profile; and blood pressure data associated with the first digital profile.

Furthermore, the first computing input comprises one or more of: identifier data associated with the first digital profile; demographic data associated with the first digital profile; response data associated with the first digital profile; wellness data associated with the first digital profile; symptoms data associated with the first digital profile; treatment data associated with the first digital profile; medication data associated with the first digital profile; health history data associated with the first digital profile; procedure data associated with the first digital profile; sleep data associated with first digital profile; and composition use data associated with the first digital profile.

It is appreciated that the above method can further comprise generating a second synthesis protocol associated with the first digital profile or a second digital profile based on the first computing input or a second computing input, wherein: the second synthesis protocol is comprised in a second file or document including at least one of: a second dosing procedure of the first composition associated with the first digital profile or a second composition associated with the first digital profile or the second digital profile; a second unidimensional or multidimensional digital representation of the first composition or a second composition; a second dosage amount of the first composition or the second composition; a second method of delivery of the first composition or the second composition; second response data associated with the second method of delivery of the first composition or the second composition; and second safety data indicating precautionary measures before, during, or after applying the second method of delivery of the first composition or the second composition.

It is further appreciated that the above method further comprises transmitting the second file or document within which is comprised the second synthesis protocol, to at least one of: the first computing device configured to visualize or display the second synthesis protocol or a second computing device configured to visualize the second synthesis protocol; the first dosing control system or a second dosing control system configured to implement the first dosing procedure and thereby generate the second composition; and the first delivery control system or a second delivery control system configured to implement or initiate the second method of delivery of the second composition into a biological system associated with the first digital profile or the second digital profile.

In some embodiments, the first synthesis protocol or the second synthesis protocol comprises a customized cannabinoid treatment protocol for one or more of the first digital profile or the second digital profile based on the first computing input or the second computing input.

Additionally, the first composition or the second composition can comprise a Tetrahydrocannabinol (THC) composition.

Moreover, the above method can further comprise predicting, based on the first synthesis protocol or the second synthesis protocol, a third synthesis protocol associated with a third computing input linked to the first digital profile or the second digital profile or a third digital profile, such that the predicting is based on a machine learning engine trained using: the synthesizer computing matrix; the first synthesis protocol; and the second synthesis protocol.

According to one embodiment, the above method further comprises: mapping, based on the synthesizer computing matrix, first synthesized data derived from the synthesizing or combining to a first index comprised in an index set; and generating, using the one or more computing device processors and based on the first index comprised in the index set, the first synthesis protocol.

It is appreciated that the index set comprises a multi-dimensional scale including one or more of: quantitative data values linked to a plurality of synthesized data including the first synthesized data or second synthesized data that is generated based on a second computing input that is different from the first computing input; and qualitative data values characterizing range information for each data value comprised in the quantitative data values linked to the plurality of synthesized data.

It is further appreciated that the range information comprises: a first value range indicating low composition effects data; a second value range indicating medium composition effects data; a third value range indicating optimal composition effects data; a fourth value range indicating high composition effects data; and a fifth value range indicating severe composition effects data.

In some cases, the index set is statistically Normally distributed or comprises a scaled Normal distribution.

### BRIEF DESCRIPTION OF THE DRAWINGS

The disclosure is illustrated by way of example, and not by way of limitation in the figures of the accompanying drawings in which like reference numerals are used to refer to similar elements. It is emphasized that various features may not be drawn to scale and the dimensions of various features may be arbitrarily increased or reduced for clarity of discussion. Further, some components may be omitted in certain figures for clarity of discussion.

FIG. 1 is a high level diagram of an exemplary system indicting an implementation of a multi-application network, in accordance with some embodiments of this disclosure.

FIGS. 2 and 3 illustrate exemplary functional and system diagrams, respectively, of a computing environment for implementing a digital assistant in a multi-application network.

FIG. 4 shows a multi-dimensional visualization indicating a scale associated with an assessment tool provided in this disclosure.

FIG. 5 provides an exemplary workflow for methods, systems, and computer program products that generate a care plan for cannabinoid treatment operations.

FIGS. 6A, 6B, and 6C provide exemplary workflows for methods, systems, and computer program products for generating a synthesis protocol associated with a digital profile.

FIG. 7 provides a rendition of data elements associated with the disclosed synthesizer computing matrix according to some embodiments.

### DETAILED DESCRIPTION

### Overview

The disclosed methods and systems provide a tool (e.g., a computational tool) that can be used in a variety of diverse settings or scenarios associated with, among other things, dosing of compositions (e.g., cannabinoid or Tetrahydrocannabinol (THC) compositions). For example, the disclosed tool can be used in scenarios where compositions are delivered including: in-patient acute care settings; and/or in-patient intensive care settings; and/or outpatient clinical settings; and/or nursing facility settings; and/or long-term acute care facilities; and/or research studies settings; and/or home care settings; and/or hospice care settings; and/or palliative care settings; and/or settings/platforms that rely on adaptive or machine learning (ML) or artificial intelligence models to optimize determining processes that deliver compositions (e.g., cannabis or THC compositions) to biological systems associated with user profiles. The settings/or platforms, in such cases, for example, can include a computing platform or other botanical care plan platforms that can include applications (e.g., medical applications with associated databases) and/or plug-in-based technologies or API-based systems that are communicatively coupled to third-party applications and/or website systems.

According to one embodiment, managing composition (e.g., THC and/or cannabis composition) titration for symptom relief using the disclosed methods and systems involves a structured approach that addresses underlying endocannabinoid imbalances to determine accurate dosage associated with composition titration. The disclosed methods and systems beneficially enable determining a, route for administering the accurate dosage as well as determine the cannabinoid and/or terpene combinations within a generated therapeutic index to provide symptom relief for medical conditions such as chronic pain, neuropathic pain, anxiety, spasticity, insomnia, depression, headaches, nausea, appetite stimulation, attention deficit hyperactivity disorder (ADHD), and stress. According to one embodiment, the disclosed methods and systems advantageously facilitate minimizing side effects of associated with taking compositions (e.g., THC and/or cannabis compositions) based on the accurate dosage determinations referenced above.

### Network Environment

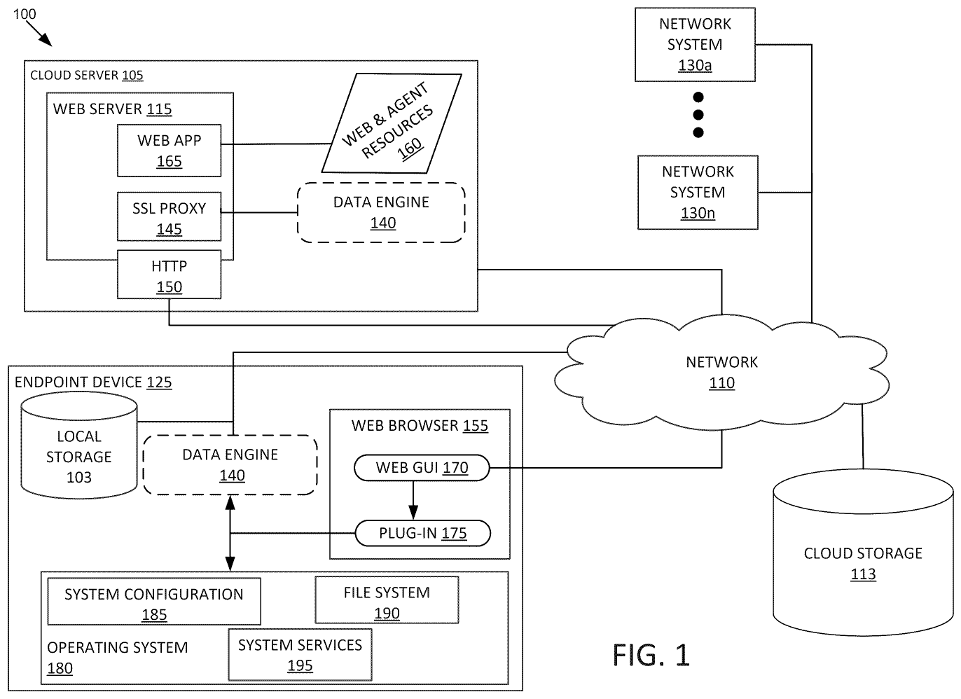

Illustrated in FIG. 1 is a high level diagram of an exemplary system 100 indicating an implementation of a network within which the disclosed techniques can be implemented. In the illustrated implementation, the system 100 may include a cloud server 105 communicatively coupled to a plurality of network systems 130a . . . 130n via a network 110. The system 100 may also include an endpoint device 125 and cloud storage 113 communicatively coupled via the network 110. While a single cloud server 105 and a single endpoint device 125 are illustrated, the disclosed principles and techniques could be expanded to include multiple cloud servers, multiple endpoint devices, and multiple cloud storage devices.

According to one embodiment, the cloud server 105 may include a computing device such as a mainframe server, a content server, a communication server, a laptop computer, a desktop computer, a handheld computing device, a smart phone, a wearable computing device, a tablet computing device, a virtual machine, a mobile computing device, a cloud-based computing system, a cloud-based service computing system, and/or the like. The cloud server 105 may include a plurality of computing devices configured to communicate with one another and/or implement the techniques described herein.

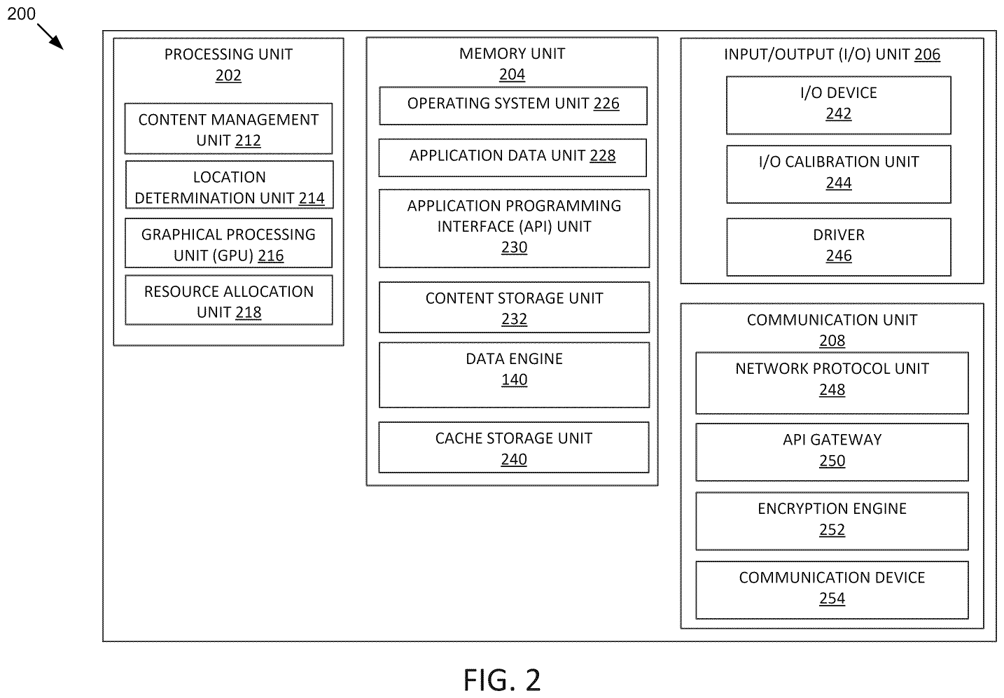

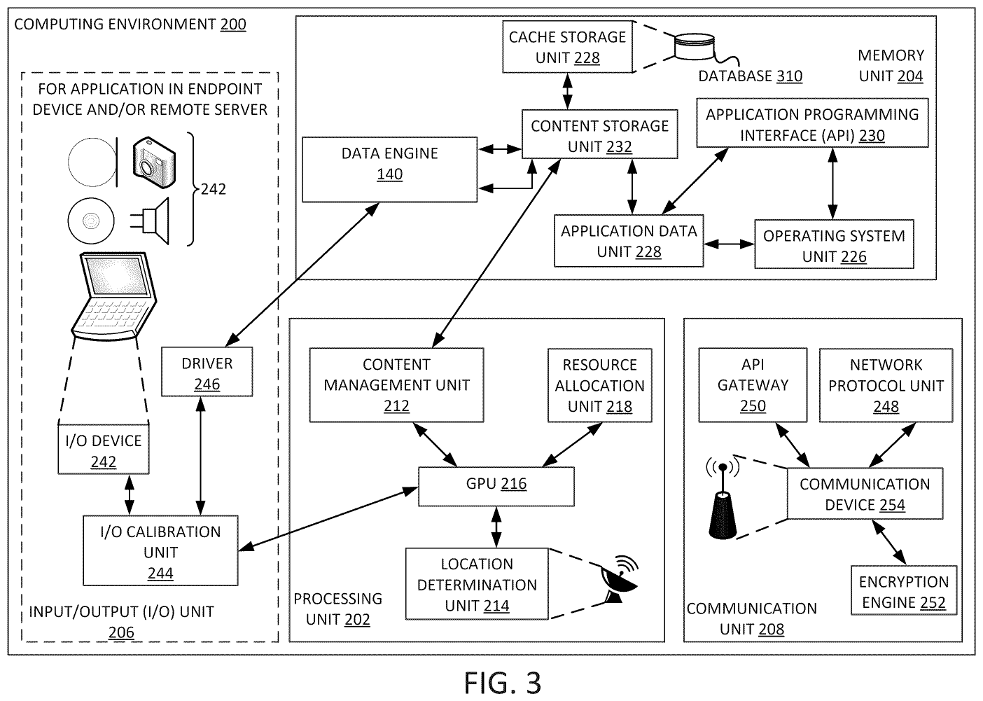

The cloud server 105 may include various elements of a computing environment as described in association with the computing environment 200 of FIGS. 2 and 3. For example, the cloud server 105 may include a processing unit 202, a memory unit 204, an input/output (I/O) unit 206, and/or a communication unit 208 which are discussed in association with FIGS. 2 and 3. Furthermore, the cloud server 105 may include subunits and/or other computing modules that perform computing operations associated with titration (e.g., cannabinoid titration operations). The cloud server 105 may be locally or remotely operated relative to a user of the disclosed system.

Turning back to FIG. 1, the cloud server 105 may include a web server 115, a data engine 140, and a web and agent resources 160. The web server 115, the data engine 140 and the web and agent resources 160 may be coupled to each other and to the network 110 via one or more signal lines. The one or more signal lines may comprise wired and/or wireless connections.

The web server 115 may include a secure socket layer (SSL) proxy 145 for establishing HTTP-based connectivity 150 between the cloud server 105 and other devices or systems coupled to the network 110. Other forms of secure connection techniques, such as encryption, may be employed on the web server 115 and across other systems coupled to the network 110. Additionally, the web server 115 may deliver data artifacts (e.g., binary code, instructions, data, etc.) to the data engine 140 either directly via the SSL proxy 145 and/or via the network 110. Additionally, the web and agent resources 160 of the cloud server 105 may be provided to the endpoint device 125 via the web app 165 on the web server 115. The web and agent resources 160 may be used to render a web-based graphical user interface (GUI) 170 via the browser 155 running on the endpoint device 125.

The data engine 140 may either be implemented on the cloud server 105 and/or on the endpoint device 125 and/or at least one of the plurality of network systems 130a . . . 130n. The data engine 140 may include one or more instructions or computer logic that are executed by one or more processors such as the processors discussed in association with FIGS. 2 and 3. In particular, the data engine 140 can facilitate executing the processing procedures, methods, techniques, and workflows provided in this disclosure.

According to one embodiment, the disclosed methods and systems comprise an iterative refinement of one or more data models (e.g., learning model, large language model, or a cannabinoid titration model) associated with system 100 via feedback loops executed by one or more computing device processors and/or through other control devices or control mechanisms that make determinations regarding optimizing a given computing action, computing template, or computing model.

In some embodiments, the data engine 140 may access an operating system 180 of the endpoint device 125 in order to execute the disclosed techniques on the endpoint device 125 and/or on the cloud server 105 and/or on at least one of the plurality of network systems 130a . . . 130n. For instance, the data engine 140 may gain access into the operating system 180 including the system configuration module 185, the file system 190, and the system services module 195 in order to execute computing operations associated with titration (e.g., cannabinoid titration operations). The plug-in 175 of the web browser 155 may enable download computing operations needed that facilitate executing computing operations associated with the operating system 180, and/or the data engine 140, and/or other applications running on the endpoint device 125 or the cloud server 105 or at least one of the plurality of network systems 130a . . . 130n.

The network 110 may include a plurality of networks. For instance, the network 110 may include a wired and/or wireless communication network that facilitates communication between the cloud server 105, the cloud storage 113, the plurality of network systems 130a . . . 130n, and the endpoint device 125. The network 110, in some instances, may include an Ethernet network, a cellular network, a computer network, the Internet, a wireless fidelity (Wi-Fi) network, a light fidelity (Li-Fi) network, a Bluetooth network, a radio frequency identification (RFID) network, a near-field communication (NFC) network, a laser-based network, a 5G network, and/or the like.

The plurality of network systems 130a . . . 130n may include one or more computing devices or servers, sensors, medical devices, medical systems or services, and/or applications that can be accessed by the cloud server 105 and/or the endpoint device 125 and/or the cloud storage 113 and/or the plurality of network systems 130a . . . 130n via the network 110. In one embodiment, the plurality of network systems 130a . . . 130n comprise third-party systems, applications, and/or computing services and/or composition (e.g., cannabinoid or THC composition) analysis computing systems or composition computing systems (e.g., vendor computing systems) that are native or non-native to either the cloud server 105 and/or the endpoint device 125. The third-party systems, applications, and/or services, for example, may facilitate executing one or more computing operations associated with cannabinoid titration based on specific attributes and/or properties associated with a biological system or a user as further discussed below.

Turning back to FIG. 1, the cloud storage 113 may comprise one or more storage devices that store data, information, and instructions used by the cloud server 105 and/or the endpoint device 125 and/or the plurality of network systems 130a . . . 130n. The stored information may include information about users/biological systems (e.g., a digital profile associated with the biological systems), information about data models (e.g., learning model, cannabinoid titration model, an artificial intelligence model, etc.), cannabinoid titration data, information about analysis operations executed by the data engine 140, etc. In one embodiment, the one or more storage devices mentioned above in association with the cloud storage 113 can be non-volatile memory or similar permanent storage device and media. For example, the one or more storage devices may include a hard disk drive, a CD-ROM device, a DVD-ROM device, a DVD-RAM device, a DVD-RW device, a flash memory device, solid state media, or some other mass storage device known in the art for storing information on a more permanent basis. While the cloud storage 113 is shown as being coupled to the cloud server 105 and the endpoint device 125 via the network 110, the data in the cloud storage 113 may be replicated, in some embodiments, on the cloud server 105 and/or the endpoint device 125. That is to say that a local copy of the data in the cloud storage 113 may be stored on the cloud server 105 and/or on the endpoint device 125 and/or on one or more of the plurality of network systems 130a . . . 130n. This local copy may be synched with the cloud storage 113 so that when there are any changes to the information in the cloud storage 113, the local copy on either the cloud server 105 or on the endpoint device 125 or on the one or more of the plurality of network systems 130a . . . 130n is also similarly updated or synched in real-time or in near-real-time to be consistent with the information in the cloud storage 113 and vice versa.

Turning back to FIG. 1, the endpoint device 125 may be a handheld computing device, a smart phone, a tablet, a laptop computer, a desktop computer, a personal digital assistant (PDA), a smart device, a wearable device, a biometric device, a medical sensor, a computer server, a virtual server, a virtual machine, a mobile device, and/or a communication server. In some embodiments, the endpoint device 125 may include a plurality of computing devices configured to communicate with one another and/or implement the techniques described in this disclosure. For example, the data engine 140 may be used to implement the disclosed techniques.

The local storage 103, shown in association with the endpoint device 125, may include one or more storage devices that store data, information, and instructions used by the endpoint device 125 and/or other devices coupled to the network 110. The stored information may include various logs/records or event files, cannabinoid titration data, security event data, etc. The one or more storage devices discussed above in association with the local database 103 can be non-volatile memory or similar permanent storage device and media. For example, the one or more storage devices may include a hard disk drive, a floppy disk drive, a CD-ROM device, a DVD-ROM device, a DVD-RAM device, a DVD-RW device, a flash memory device, solid state media, or some other mass storage device known in the art for storing information on a more permanent basis.

The other elements of the endpoint device 125 are discussed in association with the computing environment 200 of FIGS. 2 and 3. For example, elements such as a processing unit 202, a memory unit 204, an input/output (I/O) unit 206, and/or a communication unit 208 may execute one or more of the modules of endpoint device 125 and/or one or more elements of the cloud server 105 shown in FIG. 1. The endpoint device 125 may also include subunits and/or other computing instances as provided in this disclosure for performing operations associated with a digital data object (e.g., digital representation of one or more cannabinoid products) and/or the multi-application network.

FIGS. 2 and 3 illustrate exemplary functional and system diagrams of a computing environment 200, according to some embodiments of this disclosure, for implementing titration computing operations (e.g., cannabinoid titration computing operations). Specifically, FIG. 2 provides a functional block diagram of the computing environment 200, whereas FIG. 3 provides a detailed system diagram of the computing environment 200.

As seen in FIGS. 2 and 3, the computing environment 200 may include a processing unit 202, a memory unit 204, an I/O unit 206, and a communication unit 208. The processing unit 202, the memory unit 204, the I/O unit 206, and the communication unit 208 may include one or more subunits for performing operations described in this disclosure. Additionally, each unit and/or subunit may be operatively and/or otherwise communicatively coupled with each other and to the network 110. The computing environment 200 may be implemented on general-purpose hardware and/or specifically purposed hardware as the case may be. Importantly, the computing environment 200 and any units and/or subunits of FIGS. 2 and/or 3 may be included in one or more elements of system 100 as described in association with FIG. 1. For example, one or more elements (e.g., units and/or subunits) of the computing environment 200 may be included in the cloud server 105 and/or the endpoint device 125 and/or the plurality of network systems 130a . . . 130n.

The processing unit 202 may control one or more of the memory unit 204, the I/O unit 206, and the communication unit 208 of the computing environment 200, as well as any included subunits, elements, components, devices, and/or functions performed by the memory unit 204, I/O unit 206, and the communication unit 208. The described sub-elements of the computing environment 200 may also be included in similar fashion in any of the other units and/or devices included in the system 100 of FIG. 1. Additionally, any actions described herein as being performed by a processor may be taken by the processing unit 202 of FIGS. 2 and 3 alone and/or by the processing unit 202 in conjunction with one or more additional processors, units, subunits, elements, components, devices, and/or the like. Further, while one processing unit 202 may be shown in FIGS. 2 and 3, multiple processing units may be present and/or otherwise included in the computing environment 200 or elsewhere in the overall system (e.g., system 100 of FIG. 1). Thus, while instructions may be described as being executed by the processing unit 202 (and/or various subunits of the processing unit 202), the instructions may be executed simultaneously or serially by one or multiple processing units 202 on one or more devices coupled to the network 110.

In some embodiments, the processing unit 202 may be implemented as one or more computer processing unit (CPU) chips and/or graphical processing unit (GPU) chips and may include a hardware device capable of executing computer instructions. The processing unit 202 may execute instructions, codes, computer programs, and/or scripts. The instructions, codes, computer programs, and/or scripts may be received from and/or stored in the memory unit 204, the I/O unit 206, the communication unit 208, subunits, and/or elements of the aforementioned units, other devices, and/or computing environments, and/or the like.

In some embodiments, the processing unit 202 may include, among other elements, subunits such as a content management unit 212, a location determination unit 214, a graphical processing unit (GPU) 216, and a resource allocation unit 218. Each of the aforementioned subunits of the processing unit 202 may be communicatively and/or otherwise operably coupled with each other.

The content management unit 212 may facilitate generation, modification, analysis, transmission, and/or presentation of content. Content may be file content, cannabinoid titration data content, content associated with one or more systems of FIG. 1, media content, security event content, or any combination thereof. In some instances, content on which the content management unit 212 may operate includes device information, user interface data, image data, text data, themes, user/biological system data comprised in a digital profile associated with the biological system, medical data, sensor data, audio data or audio files, video data or video files, documents, and/or the like. Additionally, the content management unit 212 may control the audio-visual environment and/or appearance of application data during execution of various processes (e.g., via web GUI 170 at the endpoint device 125). In some embodiments, the content management unit 212 may interface with a third-party content server (e.g., third-party content server associated with the plurality of network systems 130a . . . 130n), and/or specific memory locations for execution of its operations.

The location determination unit 214 may facilitate detection, generation, modification, analysis, transmission, and/or presentation of location information. Location information may include global positioning system (GPS) coordinates, an internet protocol (IP) address, a media access control (MAC) address, geolocation information, a port number, a server number, a proxy name and/or number, device information (e.g., a serial number), an address, a zip code, and/or the like. In some embodiments, the location determination unit 214 may include various sensors, radar, and/or other specifically purposed hardware elements for the location determination unit 214 to acquire, measure, and/or otherwise transform location information.

The GPU 216 may facilitate generation, modification, analysis, processing, transmission, and/or presentation of content described above, as well as any data described herein. In some embodiments, the GPU 216 may be used to render content for presentation on a computing device (e.g., via web GUI 170 at the endpoint device 125). The GPU 216 may also include multiple GPUs and therefore may be configured to perform and/or execute multiple processes in parallel.

The resource allocation unit 218 may facilitate determination, monitoring, analysis, and/or allocation of computing resources throughout the computing environment 200 and/or other computing environments. For example, the computing environment may facilitate processing or analyzing a high volume of data (e.g., data associated with composition (e.g., cannabis or THC composition) titration computing operations and/or data associated with a digital data object associated with compositions (e.g., cannabis or THC compositions) titration computing operations). As such, computing resources of the computing environment 200 used by the processing unit 202, the memory unit 204, the I/O unit 206, and/or the communication unit 208 (and/or any subunit of the aforementioned units) such as processing power, data storage space, network bandwidth, and/or the like may be in high demand at various times during execution of one or more computing operations disclosed. Accordingly, the resource allocation unit 218 may include sensors and/or other specially purposed hardware for monitoring performance of each unit and/or subunit of the computing environment 200, as well as hardware for responding to the computing resource needs of each unit and/or subunit. In some embodiments, the resource allocation unit 218 may use computing resources of a second computing environment separate and distinct from the computing environment 200 to facilitate a desired operation. For example, the resource allocation unit 218 may determine a number of simultaneous computing processes and/or requests for execution by one or more computing environments. The resource allocation unit 218 may also determine that the number of simultaneous computing processes and/or requests meet and/or exceed a predetermined threshold value. Based on this determination, the resource allocation unit 218 may determine an amount of additional computing resources (e.g., processing power, storage space of a particular non-transitory computer-readable memory medium, network bandwidth, and/or the like) required by the processing unit 202, the memory unit 204, the I/O unit 206, the communication unit 208, and/or any subunit of the aforementioned units for safe and efficient operation of the computing environment while supporting the number of simultaneous computing processes and/or requests. The resource allocation unit 218 may then retrieve, transmit, control, allocate, and/or otherwise distribute determined amount(s) of computing resources to each element (e.g., unit and/or subunit) of the computing environment 200 and/or another computing environment.

The memory unit 204 may be used for storing, recalling, receiving, transmitting, and/or accessing various files and/or data during operation of computing environment 200. For example, memory unit 204 may be used for storing, recalling, and/or updating cannabinoid titration data and/or biological system or user data associated with, resulting from, and/or generated by any unit, or combination of units and/or subunits of the computing environment 200. In some embodiments, the memory unit 204 may store instructions, code, and/or data that may be executed by the processing unit 202. For instance, the memory unit 204 may store code that execute operations associated with one or more units and/or one or more subunits of the computing environment 200. For example, the memory unit may store code for the processing unit 202, the I/O unit 206, the communication unit 208, and for itself.

Memory unit 204 may include various types of data storage media such as solid state storage media, hard disk storage media, virtual storage media, and/or the like. Memory unit 204 may include dedicated hardware elements such as hard drives and/or servers, as well as software elements such as cloud-based storage drives. In some implementations, memory unit 204 may be a random access memory (RAM) device, a dynamic random access memory (DRAM) device, a static random access memory (SRAM) device, flash memory, read only memory (ROM) device, and/or various forms of secondary storage. The RAM device may be used to store volatile data and/or to store instructions that may be executed by the processing unit 202. For example, the instructions stored by the RAM device may be a command, a current operating state of computing environment 200, an intended operating state of computing environment 200, and/or the like. As a further example, data stored in the RAM device of memory unit 204 may include instructions related to various methods and/or functionalities described herein. The ROM device may be a non-volatile memory device that may have a smaller memory capacity than the memory capacity of a secondary storage. The ROM device may be used to store instructions and/or data that may be read during execution of computer instructions. In some embodiments, access to both the RAM device and ROM device may be faster to access than the secondary storage.

Secondary storage may comprise one or more disk drives and/or tape drives and may be used for non-volatile storage of data or as an over-flow data storage device if the RAM device is not large enough to hold all working data. Secondary storage may be used to store programs that may be loaded into the RAM device when such programs are selected for execution. In some embodiments, the memory unit 204 may include one or more databases 310 (shown in FIG. 3) for storing any data described herein. For example, depending on the implementation, the one or more databases may be used as the local record repository 103 of the endpoint device discussed with reference to FIG. 1. Additionally, or alternatively, one or more secondary databases (e.g., the cloud storage 113 discussed with reference to FIG. 1) located remotely from computing environment 200 may be used and/or accessed by memory unit 204. In some embodiments, memory unit 204 and/or its subunits may be local to the cloud server 105 and/or the endpoint device 125 and/or remotely located in relation to the cloud server 105 and/or the endpoint device 125.

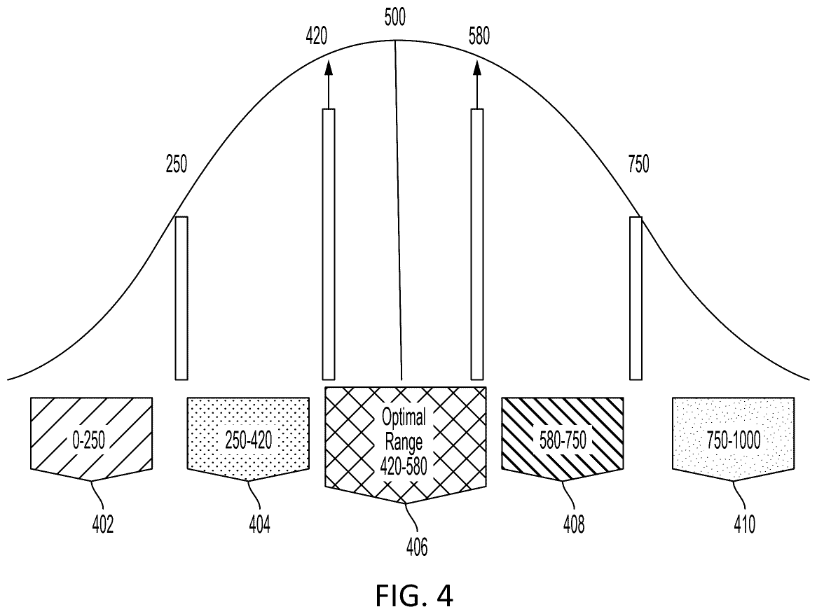

Turning back to FIG. 2, the memory unit 204 may include subunits such as an operating system unit 226, an application data unit 228, an application programming interface (API) unit 230, a content storage unit 232, data engine 140, and a cache storage unit 240. Each of the aforementioned subunits of the memory unit 204 may be communicatively and/or otherwise operably coupled with each other and other units and/or subunits of the computing environment 200. It is also noted that the memory unit 204 may include other modules, instructions, or code that facilitate the execution of the techniques described. For instance, the memory unit 204 may include one or more modules such as a data engine discussed in association with FIG. 4.

The operating system unit 226 may facilitate deployment, storage, access, execution, and/or utilization of an operating system utilized by computing environment 200 and/or any other computing environment described herein. In some embodiments, operating system unit 226 may include various hardware and/or software elements that serve as a structural framework for processing unit 202 to execute various operations described herein. Operating system unit 226 may further store various pieces of information and/or data associated with the operation of the operating system and/or computing environment 200 as a whole, such as a status of computing resources (e.g., processing power, memory availability, resource utilization, and/or the like), runtime information, modules to direct execution of operations described herein, user permissions, security credentials, and/or the like.

The application data unit 228 may facilitate deployment, storage, access, execution, and/or use of an application used by computing environment 200 and/or any other computing environment described herein. For example, the endpoint device 125 may be required to download, install, access, and/or otherwise use a software application (e.g., web application 165) to facilitate titration computing operations (e.g., cannabinoid titration computing operations). As such, the application data unit 228 may store any information and/or data associated with an application associated with executing the disclosed techniques. The application data unit 228 may further store various pieces of information and/or data associated with the operation of an application and/or computing environment 200 as a whole, such as status of computing resources (e.g., processing power, memory availability, resource utilization, and/or the like), runtime information, user interfaces, modules to direct execution of operations described herein, user permissions, security credentials, and/or the like.

The API unit 230 may facilitate deployment, storage, access, execution, and/or use of information associated with APIs of computing environment 200 and/or any other computing environment described herein. For example, computing environment 200 may include one or more APIs for various devices, applications, units, subunits, elements, and/or other computing environments to communicate with each other and/or utilize the same data. Accordingly, API unit 230 may include API databases containing information that may be accessed and/or used by applications, units, subunits, elements, and/or operating systems of other devices and/or computing environments. In some embodiments, each API database may be associated with a customized physical circuit included in memory unit 204 and/or API unit 230. Additionally, each API database may be public and/or private, and so authentication credentials may be required to access information in an API database. In some embodiments, the API unit 230 may enable the cloud server 105 and the endpoint device 125 to communicate with each other. It is appreciated that the API unit 230 may facilitate accessing, using the data engine 140, one or more applications or services on the cloud server 105 and/or the plurality of network systems 130a . . . 130n.

The content storage unit 232 may facilitate deployment, storage, access, and/or use of information associated with performance of implementing operations associated with system 100. In some embodiments, content storage unit 232 may communicate with content management unit 212 to receive and/or transmit content files (e.g., media content, digital request data object content, command content, input content, registration object content, etc.).

As previously discussed, the data engine 140 facilitates executing the processing procedures, methods, techniques, and workflows provided in this disclosure. In particular, the data engine 140 may be configured to execute computing operations associated with the disclosed methods, systems/apparatuses, and computer program products.

The cache storage unit 240 may facilitate short-term deployment, storage, access, analysis, and/or utilization of data. In some embodiments, cache storage unit 240 may serve as a short-term storage location for data so that the data stored in cache storage unit 240 may be accessed quickly. In some instances, cache storage unit 240 may include RAM devices and/or other storage media types for quick recall of stored data. Cache storage unit 240 may include a partitioned portion of storage media included in memory unit 204.

The I/O unit 206 may include hardware and/or software elements for the computing environment 200 to receive, transmit, and/or present information useful for performing the disclosed processes. For example, elements of the I/O unit 206 may be used to receive input from a user of the endpoint device 125. As described herein, I/O unit 206 may include subunits such as an I/O device 242, an I/O calibration unit 244, and/or driver 246.

The I/O device 242 may facilitate the receipt, transmission, processing, presentation, display, input, and/or output of information as a result of executed processes described herein. In some embodiments, the I/O device 242 may include a plurality of I/O devices. In some embodiments, I/O device 242 may include a variety of elements that enable a user to interface with computing environment 200. For example, I/O device 242 may include a keyboard, a touchscreen, a button, a sensor, a biometric scanner, a laser, a microphone, a camera, and/or another element for receiving and/or collecting input from a user. Additionally, and/or alternatively, I/O device 242 may include a display, a screen, a sensor, a vibration mechanism, a light emitting diode (LED), a speaker, a radio frequency identification (RFID) scanner, and/or another element for presenting and/or otherwise outputting data to a user. In some embodiments, the I/O device 242 may communicate with one or more elements of processing unit 202 and/or memory unit 204 to execute operations associated with the disclosed techniques and systems.

The I/O calibration unit 244 may facilitate the calibration of the I/O device 242. For example. I/O calibration unit 244 may detect and/or determine one or more settings of I/O device 242, and then adjust and/or modify settings so that the I/O device 242 may operate more efficiently. In some embodiments, I/O calibration unit 244 may use a driver 246 (or multiple drivers) to calibrate I/O device 242. For example, the driver 246 may include software that is to be installed by I/O calibration unit 244 so that an element of computing environment 200 (or an element of another computing environment) may recognize and/or integrate with I/O device 242 for the processes described herein.

The communication unit 208 may facilitate establishment, maintenance, monitoring, and/or termination of communications between computing environment 200 and other computing environments, third party server systems, and/or the like (e.g., between the cloud server 105 and the endpoint device 125 and or the plurality of network systems 130a . . . 130n). Communication unit 208 may also facilitate internal communications between various elements (e.g., units and/or subunits) of computing environment 200. In some embodiments, communication unit 208 may include a network protocol unit 248, an API gateway 250, an encryption engine 252, and/or a communication device 254. Communication unit 208 may include hardware and/or other software elements.

The network protocol unit 248 may facilitate establishment, maintenance, and/or termination of a communication connection for computing environment 200 by way of a network. For example, the network protocol unit 248 may detect and/or define a communication protocol required by a particular network and/or network type. Communication protocols used by the network protocol unit 248 may include Wi-Fi protocols, Li-Fi protocols, cellular data network protocols, Bluetooth® protocols, WiMAX protocols, Ethernet protocols, powerline communication (PLC) protocols, and/or the like. In some embodiments, facilitation of communication for computing environment 200 may include transforming and/or translating data from being compatible with a first communication protocol to being compatible with a second communication protocol. In some embodiments, the network protocol unit 248 may determine and/or monitor an amount of data traffic to consequently determine which particular network protocol is to be used for establishing a secure communication connection, transmitting data, and/or performing titration operations and/or other processes described herein.

The API gateway 250 may allow other devices and/or computing environments to access the API unit 230 of the memory unit 204 associated with the computing environment 200. For example, an endpoint device 125 may access the API unit 230 of the computing environment 200 via the API gateway 250. In some embodiments, the API gateway 250 may be required to validate user credentials associated with a user of the endpoint device 125 prior to providing access to the API unit 230 to a user. The API gateway 250 may include instructions for the computing environment 200 to communicate with another computing device and/or between elements of the computing environment 200.

The encryption engine 252 may facilitate translation, encryption, encoding, decryption, and/or decoding of information received, transmitted, and/or stored by the computing environment 200. Using encryption engine 252, each transmission of data may be encrypted, encoded, and/or translated for security reasons, and any received data may be encrypted, encoded, and/or translated prior to its processing and/or storage. In some embodiments, encryption engine 252 may generate an encryption key, an encoding key, a translation key, and/or the like, which may be transmitted along with any data content.

The communication device 254 may include a variety of hardware and/or software specifically purposed to facilitate communication for computing environment 200. In some embodiments, communication device 254 may include one or more radio transceivers, chips, analog front end (AFE) units, antennas, processing units, memory, other logic, and/or other components to implement communication protocols (wired or wireless) and related functionality for facilitating communication for computing environment 200. Additionally and/or alternatively, communication device 254 may include a modem, a modem bank, an Ethernet device such as a router or switch, a universal serial bus (USB) interface device, a serial interface, a token ring device, a fiber distributed data interface (FDDI) device, a wireless local area network (WLAN) device and/or device component, a radio transceiver device such as code division multiple access (CDMA) device, a global system for mobile communications (GSM) radio transceiver device, a universal mobile telecommunications system (UMTS) radio transceiver device, a long term evolution (LTE) radio transceiver device, a worldwide interoperability for microwave access (WiMAX) device, and/or another device used for communication purposes.

### Exemplary Embodiments

The disclosed approach provides a tool (e.g., a computational cannabinoid assessment tool) that effectively gauges, titrates, and/or customizes cannabinoid or THC dosages for specific biological systems or user classes or digital profiles associated therewith. According to one embodiment, the tool ingests or receives data via an input device (e.g., input computing device. The data, for example, can be automatically received based on profile data or account data associated with a biological system or a caregiver or a healthcare worker and in some cases, entered into the disclosed system by the biological system or the caregiver or the healthcare worker. For example, the user and/or biological system and/or caregiver and/or healthcare giver may provide input data to the tool such that the input data (e.g., voice data, video data, textual data, medical record data, medical data, observable biological system data, laboratory data, medical sensor/equipment data associated with a user or biological system, etc.) characterizes or otherwise describes a plurality of identifying information and/or medical information associated with the user or biological system. For example, the input data may include captured data indicating observable biological system information or user assessment information, or other medical records. In one embodiment, the observable biological system information or user assessment information comprises: a detailed description of symptoms (e.g., symptoms data and/or problematic symptoms) of a user or a biological system from unknown or known causes; and/or symptoms data associated with acute or chronic illnesses of the user or biological system; and/or side effects data (e.g., desirable or undesirable side effects data) from medications or supplements that needs to be amplified or minimized for said biological system or user; and/or element interactions data associated with the use of alternative botanical substances like THC or cannabinoid compositions.

According to one embodiment, the input data ingested by the disclosed tool includes one or more of: identifier data (e.g., biographic data including name information) of a user or biological system; demographic data including age data and/or gender data and/or race data and; substance reaction data or substance response data (e.g., allergic reaction data); health data or wellness data associated with health goals or objectives of a user or a biological system; symptoms data associated with a medical condition of the user or biological system including health condition data; and/or treatment resolution data indicating a discomfort information and/or pain estimation data associated with the user or biological system; substance or medication data that have historically assisted in alleviating and/or eliminating said health condition data and/or discomfort information for said biological system or user or other users or biological systems; medical history data or health history data of the user or biological system; data indicating history of hospital admissions of the biological system or user within a past timeframe (e.g., within the last 1 year, 2 years, 3 years, etc.); data describing reasons for said hospital admission; data indicating surgical history of the user or biological system within a past timeframe (e.g., within the last 3 years); data (e.g., medical procedure data) indicating upcoming medical procedures (e.g., surgeries, etc.) of the biological system or user; family medical history data associated with the user or biological system; data (e.g., medical condition data) indicating specific medical conditions (e.g. schizophrenia, heart disease, diabetes, or sudden cardiac death) of user classes or biological system classes to which the user or the biological system belongs; height data and weight data associated with the user or biological system; data indicating prescribed medications being taken by the user or biological system; side effect data associated with medications or substances being taken by the user or biological system; data indicating supplements (e.g., vitamins, etc.) being taken by the user or biological system; side effect data associated with taking said supplements; data indicating wellness activities including holistic treatments/practices (Qi Gong, Acupuncture, meditation, etc.) being implemented by the user with associated response data (e.g., physiological response data) generated within the user or biological system; diet data associated with the user or biological system; exercise data associated with the user or biological system; personality data associated with the user or biological system; sleep data (e.g., frequency and duration data) and effects thereof associated with sleep patterns of the user or biological system; data indicating history of cannabinoid or cannabis or CBD use of the biological system or user with associated dosage and effects information; Cannabinoids previously used by the user or biological system; data indicating types of cannabis or cannabinoid products that are currently or previously being used by the user or biological system together with attendant modes of delivery, flavors, etc.; data indicating medical and/or supplement and/or cannabinoid preferences of the user or biological system; and data indicating medical cards or other identifiers that provide access to cannabinoid products to the user or biological system.

According to one embodiment, a data schema (e.g., scoring schema) is leveraged to generate a computing parameter (e.g., a quantitative score or weight parameter and/or qualitative characterization parameter consolidated into the disclosed therapeutic index) that indicates a dosing category to which a user or biological system belongs. Exemplary implementations include newly generating the computing parameter for a user or biological system who has not previously used the tool following which an updated computing parameter may be subsequently generated based on cannabinoid use response data of the user after using the tool. In other embodiments, an estimate of the computing parameter may be generated for the user or biological system after which the tool is used to update or otherwise generate a more optimized or accurate computing parameter for the user or biological system. It is appreciated that the initial computing parameter generated with or without the use of the tool merely facilitate or serves as a guide for preparing or titrating starting cannabinoid dose ranges and recommendations of THC and cannabis products for the user or biological system.

In some embodiments, a review (e.g., literature review) of research data and/or other biological system or user response data may be analyzed based on the generated computing parameter to support or otherwise inform or validate current dosing strategy data generated based on the computing parameter or therapeutic index for the user or biological system. This dosing strategy may be used to generate support data for the disclosed titration operation based on the generated parameter. The support data, for example, may be factored into: product recommendations (e.g., cannabinoid product recommendations) based on biological system or user preference data (e.g., derived from the input data discussed above); nursing therapeutics and care work data; biological system safety data; drug-drug interactions data; etc. In some embodiment, the computing parameter discussed above may be generated using subjective data (e.g., data from a user or biological system) and/or objective data (e.g., data from a caregiver, a doctor, or a nurse).

Furthermore, product recommendations and/or cannabinoid dosages in a care plan or a cannabinoid treatment protocol for a user may be provided based on the generated computing parameter. For example, the care plan may comprise a botanical care plan that provides diagnosis data (e.g., nursing diagnosis data), goal setting data, safety precautions data, risk reduction data, rationale data, and additional supplemental interventions data (e.g., nursing/user interventions data), study citations data that drive or otherwise inform the use of the disclosed tool. In some cases, the disclosed tool: enables review of the care plan (e.g., botanical care plan); provides an overview of care education; and teaches/reviews how to use the disclosed tool by users or biological systems, medical teams, and caregivers. In some embodiments, the disclosed tool may be used to guide multiple follow-up dosing and titration operations to guide, in real-time or near real-time, residence nurse operations, and/or other healthcare worker operations, and/or some other caregiver operations associated with optimally titrating and/or accurately preparing effective cannabinoid doses for a user or biological system. In some embodiments, such follow-up dosing and titration operations may range from 1-10 times in frequency, and/or may continue in perpetuity.

In scenarios where a user or biological system brings their own supply of cannabinoid doses and/or products to a health facility or has same (e.g., based on prescription) at place of residence, said cannabinoid doses or products may be securely stored in a pharmaceutical bin for the user or biological system. In the case of a healthcare facility, an in-patient healthcare worker (e.g., a licensed practical nurse (LPN), a registered nurse (RN), a physician assistant (PA), an advanced practice nurse (APN), a doctor of medicine (MD), a Doctor of Osteopathic Medicine (DO), and a respiratory therapist) may use the tool to generate the computing parameter discussed above to guide the dosing and delivery of the biological system's own cannabinoid doses or products and/or supplement same using other cannabinoid doses and products based on the generated computing parameter. The disclosed tool may be used in other settings including home healthcare settings, home hospice settings, doctors' offices and dentists' offices, medical spas, surgical centers, school nurses' offices within jurisdictions with legalized use of cannabinoids, senior day programs, halfway houses, group homes, and other types of unaccounted for outpatient centers.

According to one embodiment, the disclosed tool comprises a tetrahydrocannabinol physiologic assessment tool (TPAT) (e.g., a physiological plant medicine tool) which has an attendant scale that is similar in shape to, or substantially corresponds to a Gaussian or Normal distribution curve or a bell shaped curve. In particular, the scale or the bell shaped curve comprises multi-dimensional data that is scaled to have quantitative data values (e.g., values between 0 to 1,000 with 500 as the midpoint) or qualitative data values (e.g., optimal range data) in a first dimension that correspond to qualitative or quantitative data values in a second dimension as depicted in FIG. 4. According to one embodiment, the qualitative values or qualitative data values in the first dimension comprises one or more of: a first value range 402 indicating low cannabinoid effects data; a second value range 404 indicating medium cannabinoid effects data; a third value range 406 indicating optimal cannabinoid effects data, a fourth value range 408 indicating high cannabinoid effects data; and a fifth value range 410 indicating severe cannabinoid effects data. In exemplary implementations, the quantitative values are split or otherwise divided into the following distinct sections comprising an index set: 0-250, 250-420, 420-580, 580-750, 750-1000. It is appreciated that each of the distinct sections can encompass or indicate: a variety of qualitative objective and/or subjective biological system data or user data; and/or symptoms data associated with the biological system or user; and/or other health data (e.g., vital signs information) associated with the biological system or user. In one embodiment, the first dimension can be formatted to incorporate a multi-point (e.g., 7-point) scale for symptom control ranging from 0 (e.g., indicating no relief and adverse effects only) to 6 (e.g., indicating complete resolution of symptoms).

After a composition (e.g., cannabis or THC composition) is administered to a biological system or user, various use criteria, use qualities, and/or use characteristics may be leveraged to determine or otherwise establish a set of physiological symptoms and potential side effects on the user or biological system which can in turn be used to identify the scope of therapeutic effect and any potential adverse side effects from using the composition administered to the user or biological system. In some implementations, users or biological systems as well as care providers can enter inputs into the disclosed tool such that the inputs comprise or indicate a therapeutic index which can visually reflect effects of compositions (e.g., cannabis or THC compositions) on the biological system or user in response to administering the compositions to the biological system or user. In one embodiment, the therapeutic index represents a value range for relief of symptoms and can range from anywhere between a tetrahydrocannabinol physiologic assessment tool (TPAT) score range of 150-750. Less than 150 may be considered subtherapeutic, and greater than 750 indicates that the adverse effects of a given cannabinoid dose (e.g., cannabis or THC composition dose) are too significant and that said dose should be reduced.

In some embodiments, the disclosed tool comprises an electronic or digital system that is able to receive a plurality of inputs from a user and designate a qualitative or quantitative value comprised in the aforementioned sale to guide the preparation and administration of a composition (e.g., cannabinoid composition or THC composition). In particular, the disclosed tool can guide the preparation and administration of both an initial cannabinoid dose and subsequent dose adjustments for the disclosed compositions. According to one embodiment, an artificial intelligence (AI) engine or machine learning (ML) engine associated with the disclosed tool may be trained or otherwise adapted to aggregate user or biological system data indicating a plurality of composition (e.g., THC and cannabinoid composition) dosages customized for a plurality of biological system classes or user classes. In particular, the user or biological system data may be used to train or otherwise configure learning structures of the AI or ML engine over time to enable the AI or ML engine to predict qualitative or quantitative values (e.g., therapeutic indices) for new biological system or user data. This approach beneficially informs the precise assignment of a particular biological system or user to an appropriate user or biological system class for initial or subsequent composition dosing. In some cases, the AI or ML engine beneficially validates newly computed therapeutic indices thereby enhancing the robustness and accuracy of the computed therapeutic indices.

In an exemplary implementation, the disclosed tool can be used for an initial assessment (e.g., designation of a biological system to a particular qualitative or quantitative value category comprised in the scale) at a first time (e.g., t₀) of consumption of the composition dosage for all routes of administration and thereby perform an assessment and/or collect subjective and objective data associated with the user or biological system's response to said first composition intake. The assessment may then be repeated at a second time (e.g., t₁) using the assessment tool where the second time comprises one or more of: a time of estimated peak effect of the initial composition intake; and/or a time at the end of an estimated duration of a user action or activity or biological system action or activity associated with the initial composition intake; and/or a time at a midpoint check-in for longer lasting routes of administration of compositions (e.g., cannabinoid or THC compositions) to the biological system or user; and/or a designated time (e.g., 10 minutes, 15 minutes, 30 minutes, 60 minutes, etc.) of an optional post-consumption assessment as the case may require.

According to some embodiments, the disclosed assessment tool may be based on, or may have one or more of the following associated recommended temporal intervals associated with one or more routes of administering a composition dosage together with attendant products to a user or biological system:
(i) Product: Smoking Flower
   Peak effect—15 minutes after intake
   Mid-Duration: 30 minutes after intake
   End of Duration: 2 hours after intake
(ii) Product: Vaping Flower
   Peak effect—15 minutes after intake
   Mid-Duration: 30 minutes after intake
   End of Duration—2 hours after intake
(iii) Product: Vaping Oil
   Peak effect—15 minutes after intake
   Mid-Duration: 30 minutes after intake
   End of Duration—2 hours after intake
(iv) Product: “Dabbing”—High Concentrated Oils [Like Hash, Wax, etc.]
   Peak effect—15 minutes after intake
   Mid-duration: 30 minutes after intake
   End of Duration—2 hours after intake
(v) Product: Sublingual Oil Tinctures and Sprays
   Peak effect—45 minutes after intake
   Mid-Duration—3 hours after intake Late Duration—6 hours after intake
(vi) Product: Transdermal Patches
   Peak effect—20 minutes after application Mid Duration—4 hours after intake Late Duration—10 hours after intake
(vii) Product: Ingested Solid Edible
   Peak effect—45 minutes after application
   Mid Duration—4 hours after intake
   Late Duration—10 hours after intake
(viii) Product: Ingested Liquid Edible
   Peak effect—20 minutes after application
   Mid Duration—4 hours after intake
   Late Duration—10 hours after intake
(ix) Product: Topical Creams and Lotions
   Peak effect—10 minutes after application
   Mid Duration—1 hour after intake
   Late Duration—4 hours after intake
(x) Product: Rectal Suppositories
   Peak effect—45 minutes after intake
   Mid-Duration—3 hours after intake
   Late Duration—6 hours after intake
(xi) Product: Vaginal Suppositories
   Peak effect—45 minutes after intake
   Mid-Duration—3 hours after intake
   Late Duration—6 hours after intake
(xii) Product: Eye/Ear Drops
   Peak effect—45 minutes after intake
   Mid-Duration—3 hours after intake
   Late Duration—6 hours after intake
(xiii) Product: Intravenous
   Peak—5 minutes after injection/bolus/drip
   Mid-Duration—15 minutes after injection/bolus/drip
   Late Duration—45 minutes after injection/bolus/drip

In exemplary implementations, a highest qualitative or quantitative value (e.g., a value within a zone range, a user or biological system category or class) may be determined for the user or biological system using the disclosed tool. In such cases, the highest qualitative or quantitative value may be used to adjust or otherwise customize composition doses (e.g., THC doses) based on one or more of:
(i) Zone 1: 0-250
   Increase THC dose by 50% OR
   Add other cannabinoids such as Cannabidiol (CBD) or Cannabigerol (CBG) at a 20:1 ratio of the THC dose.
(ii) Zone 2: 250-420
   Increase THC dose by 25% OR
   Add other cannabinoids such as CBD or CBG at a 20:1 ratio of the THC dose. OR
   Add additional Terpenes OR
   No changes/stay with current dose if desired.
(iii) Zone 3: 420-580
   No changes/stay with current dose.
(iv) Zone 4: 580-750
   Reduce THC dose by 25% OR
   Add other cannabinoids such as CBD or CBG at a 20:1 ratio of the THC dose. OR
   Add additional Terpenes OR
   No changes/stay with current dose if desired.
(v) Zone 5: 750-1000
   Reduce THC dose by 50%.

According to one embodiment, a user, biological system, or caregiver may wait for a first wait time (e.g., a minimum of 4-8 hours or 8-12 hours) before redosing composition dosages (e.g., oral solid/liquid edibles, vaginal/rectal suppositories, sublingual spray/oil, or eye/ear drops) after a first use of the composition dosages directed by the use of the disclosed tool before taking a new dose. In some cases, a user, biological system, or caregiver may wait for a second wait time (e.g., a minimum of 2-4 hours) before redosing compositions that are inhaled (e.g., smoking or vaping or dabbing) or used based on a transdermal method after a previous dose (e.g., first dosage intake or subsequent dosage intake).

In some embodiments, data variables collected based on computing operations associated with the disclosed tool comprise: an estimated data value or score for the biological system or user (e.g., TPAT score within the range of 0-1000); data indicating a symptom or a condition (e.g., medical condition) being treated; data indicating a Likert scale for symptom control (e.g., within a range of 0-6); heart rate data; blood pressure data; data associated with a cannabis composition being used (e.g. product and/or ingredients being used or to be administered) together with attendant attributes including cannabinoids. terpenes, strain, etc., delivered to the user or biological system; dose of the composition (e.g., THC composition) in milligram/gram of said composition; dose of other cannabinoids including CBD, CBG, and CBC in milligram/gram; route of administration of composition to the user; reported adverse side effects of said compositions or brands associated with same. In some cases, the data entered into the tool further includes: diagnosis data associated with the user or biological system; subjective and objective health symptoms data of the user or biological system; healthcare goals data of the user or biological system; age data, gender data, height data, and weight data of the user or biological system; mobility status data and/or cognitive abilities data and/or diet data of the user or biological system; exercise data of the user or biological system; health outcomes data indicating user or biological system's responses to treatment therapies and options; pharmaceutical data of medications being used by the user or biological system together with supplements data being used by the user or biological system concurrently with or separate from previous cannabis use by the user or biological system; tobacco use history data of user or biological system; illicit drug use history data of user or biological system; medical history data of user or biological system; family history data of user or biological system; and/or menstrual cycle information of the biological system.

In some embodiments, the disclosed methods and systems facilitate accurate dosing, scoring, and digitally expanding insights regarding human interactions to cannabis, routes of administration, pathophysiology, therapeutic response, drug interactions, food interactions, supplement interactions, and side effects using the disclosed tool. In particular, the disclosed tool may further leverage additional data (e.g., composition data from product databases) to more accurately or otherwise optimally designate or provide optimal values for biological systems or users whose data is submitted to the tool for assessment. For example, the additional data may include: respiratory rate data; pulse oximetry data; ejection fraction (EF) data; central venous pressure (CVP) data; mean arterial pressure (MAP) data; cardiac output (CO) data; systemic vascular resistance (SVR) data; pulmonary arterial pressure (PAP) data; pulmonary function tests (PFTs) data including forced vital capacity (FVC) data, total lung capacity data, vital capacity data, airway resistance data, residual volume data, inspiratory capacity (IC) data, forced expiratory volume (FEV1) data, FEV1/FVC ratio data. positive end expiratory pressure (PEEP) data; cortisol Level data; fasting blood sugar data; glucose data; arterial blood gas (ABG) data; venous blood gas (VBG) data; venous pH data; arterial pH data, carbon dioxide (CO2) data, oxygen (O₂) data; bicarbonate (HCO₃) data; white blood cell count (WBC) data; hemoglobin (Hbg) data; hematocrit (Het) data; Iron data, red blood cell count (RBC) data; platelets data; hemoglobin A1C (HbgA1C) data; Troponin level data; aspartate transaminase (AST) data; Alanine transaminiase (ALT) data; alkaline phosphatase (AP) data; B-type natriuretic peptide (BNP) data, atrial natriuretic peptide (ANP) data; anion gap data; creatinine (Cr) data; blood urea nitrogen (BUN) data; glomerular filtration rate (GFR) data; creatinine clearance data; renin data; aldosterone data; estrogen data; testosterone data; procalcitonin data; calcium data; magnesium data; sodium data; potassium data; erythrocyte sedimentation rate (ESR) data; urinalysis data; ACTH test data; thyroid data; stimulating hormone (TSH) data: triiodothyronine (T3) data; thyroxine (T4) data; lutenizing hormone (LH) data; follicular stimulating hormone (FSH) data; ammonia level data; estradiol data; triglycerides data; low-density lipoprotein (LDL) data; high-density lipoprotein (HDL) data; internation normalized ratio (INR) data; prothrombin time (PT) data; partial thromboplastin time (PTT) data; D-Dimer data: C-reactive protein (CRP) data; serum amyloid A data; fibrinogen data; cytokines (e.g., predominantly tumor necrosis factor alpha (TNFα) data: interleukins 1β, 6, 8, 10, 12 and their receptors' data: Interferon gamma (IFNγ) data; haptoglobin data; hepcicin data; plasma viscosity data; alpha-1 acid glycoprotein data; ceruloplasmin data; positron emission tomography (PET) scan data; neurotransmitter data including dopamine data, serotonin data, gamma-aminobutyric acid (GABA) data; electroencephalogram (EEG) frequency patterns data including frequency data indicating: beta (14-30 Hz) states, alpha (8-13 Hz) states, theta (4-7 Hz) states, and delta (1-3 Hz) states; range of motion (ROM) data; activities of daily living (ADLs) data; Ashworth scale/modified Ashworth scale results data; antinuclear antibody (ANA) data; autoantibody tests data; echocardiogram (EKG) data; pain scale (e.g., 1-10) data; Wong-Baker Pain scale data, Glascow coma scale (GSC) data; Richmond agitation scale (RASS) data; therapeutic interventions scoring system (TISS) data; acute physiology data; age and chronic health evaluation systems (APACHE) data; simplified acute physiology score (SAPS) data; physiological and operative severity score for the enumeration of mortality and morbidity (POSSUM) data; sequential organ failure assessment (SOFA) data; and EuroSCORE data including CABG data, MELD data, TEE data, and TTE.

After ingesting the aforementioned data, the disclosed tool (e.g., composition assessment tool) generates a value within a given user or biological system category or class comprised in the scale associated with the tool to drive or otherwise inform titration and/or dosing operations for composition (e.g., cannabinoid or THC composition) administration to a user.

According to one embodiment, the AI or ML engine referenced above may categorize biological systems into appropriate zones or categories for accurate titration recommendations and generation of representative TPAT scores for biological systems or users to enable avoidance of severe or adverse side effects of composition doses for biological systems and/or improve efficacy of composition doses for biological systems or users, as well as provide effective therapeutic relief to users or biological systems.

In addition, the disclosed tool may provide research data (e.g., scientific insights) about composition dosing relative to human physiology, route of administration symptoms, compositions, side effects of said compositions, and other variables that affect cannabinoid or THC use including rate of absorption, drug interactions, ingredient diversity, cannabinoid diversity, and terpene diversity. In one embodiment, one or more of the biological system or user data, research data, or other data referenced herein may be stored locally on a user or health facility storage system and/or stored in a distributed database such as a cloud database.

According to one embodiment, the disclosed tool (also referred to as assessment tool or TPAT assessment tool herein) may be used to generate and/or implement a care plan for a biological system or user. The care plan or a cannabinoid treatment protocol, for example, may comprise a document, a file, or a form on a graphical interface or a print thereof that indicates a procedure for titrating or preparing one or more cannabinoids or THC doses, and/or include one or more cannabinoid or THC compositions associated with said doses, and/or include delivery methods of said cannabinoid or THC compositions, and/or include guidelines or expected biological system or user symptoms in response to the biological system or user taking said cannabinoid or THC compositions, and/or other safety instructions for the biological system or user. According to one embodiment, the care plan includes a recommendation of a combination of one or more cannabinoid or THC compositions (e.g., CBD composition, CBN composition, THC composition, or a combination of the aforementioned compositions) that work together to enhance or improve the therapeutic effect of the cannabinoid or THC composition.

It is appreciated that, in response to providing the tool with one or more of the aforementioned input data, the tool may generate a therapeutic index (e.g., a qualitative and/or quantitative value comprised in a scale associated with the tool) which can drive the generation and implementation of the care plan. According to one embodiment, the care plan beneficially allows the control of symptoms and side-effects associated with taking one or more cannabinoid or THC compositions.

In other embodiments, the tool may be electronically linked or otherwise coupled to databases such as those discussed in association with FIG. 1 such that once the care plan is generated, the care plan includes links or display elements that can be dynamically activated to provide options or sources for selecting one or more cannabinoid or THC compositions based on the therapeutic index. For example, display elements may be tied to one or more compositions with as follows:
(i) A. Composition name and producer
   Type of administration
   Total amount in container.
   Recommended dosing rage.
   Delivery instructions
   Link 1
(ii) B. Composition name and producer
   Type of administration
   Total amount in container.
   Recommended dosing rage.
   Items in stock at
   XXXX Dispensary In Location as of a first date:
   Link 2
(iii) C. Composition name and producer
   Type of administration
   Total amount in container.
   Recommended dosing rage.
   Instructions
   Link 3
(iv) D. Composition name and producer
   Type of administration
   Total amount in container.
   Recommended dosing rage.
   Instructions
   Link 4

According to one embodiment, the therapeutic index generated by the tool may include a quantitative value in one or more user or biological system classes and which directs or otherwise recommends specific dosage instructions. For example, the one or more user or biological system classes may be comprised in one of the following ranges indicating the biological system and user classes with attendant instructions:
0-250: Increase dose.
250-420: Increase dose or continue using as is and re-evaluate.
420-580: Stay at current dose.
580-750: Decrease current dose.
>750: Decrease current dose significantly; drink water, rest, chew on citrus rind or eat something spicy.

Furthermore, the disclosed tool, according to one embodiment, may receive static biological system or user data (e.g., received by inputs from users or biological systems or caregivers) and/or dynamic biological system or user data (e.g., received directed from sensors coupled to the user or biological system or from institutions (e.g., medical laboratories) conducting tests on biological system or user biological samples) such that the static and/or dynamic data may be independently leveraged and/or leveraged in aggregate to generate a new therapeutic index or an updated therapeutic index for optimized preparation and deliver of a new dose of cannabinoid or THC composition to a user or biological system.

According to one embodiment, the disclosed tool is based on one or more computing models (e.g., therapeutic computing models) that have parameters including one or more of: a care plan parameter; biological system health history parameter; biological system lab data parameter; a physician input parameter; physiological measurement parameter derived from sensors or medical devices coupled to the biological system or user or which analyze biological system or user biological samples; cannabinoid response parameter indicating effects of one or more cannabinoid compositions on a biological system, etc. In some implementations, the assessment tool is an AI or ML tool that is trained using the one or more models to generate a care plan that can predict cannabinoid or THC dosages/compositions that is applied to specific biological system or user demographics including one or more of: biological system or user ages, biological system or user genders, biological system or user races, biological system or user health histories, etc. The AI or ML tool can also provide activity recommendation data (e.g., dosage data) for a given biological system demographic; and/or frequency data of dosage intake; and/or substances (e.g., water, food, etc.) intake data to drive activities associated with taking a specific cannabinoid dosage or composition. Moreover, the AI or ML tool can provide safety activity data (e.g., data indicating that cannabinoid or THC intake should be avoided to minimize harmful side-effects and/or impact on activities such as driving or operating a vehicle after a given dosage is taken) associated with taking a specific cannabinoid dosage or THC composition based on a given care plan.

### First Exemplary Workflow

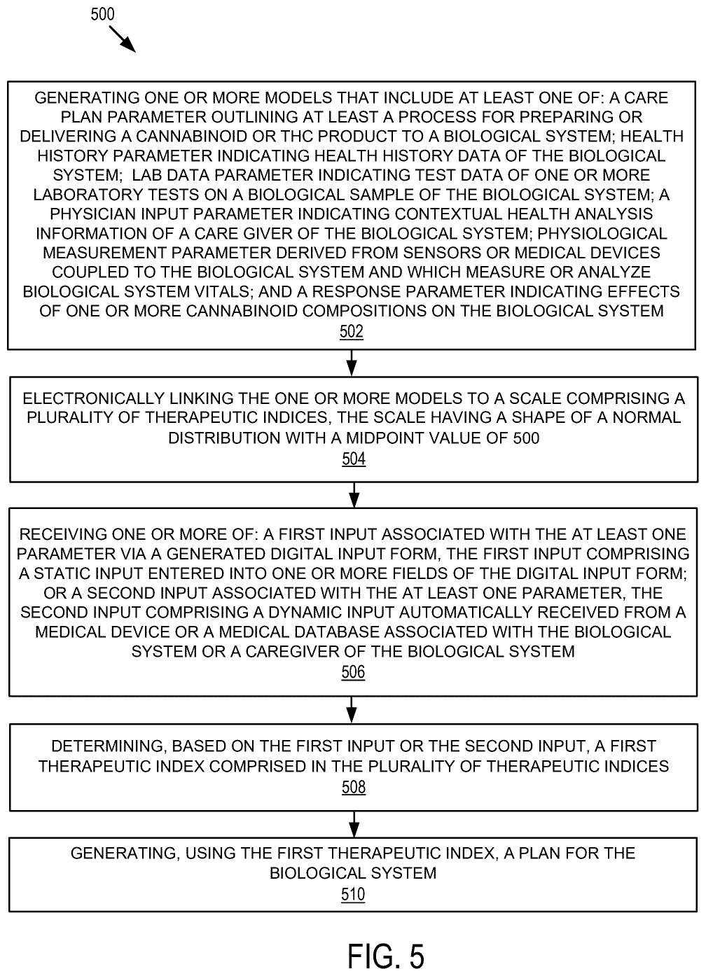

FIG. 5 provides an exemplary workflow 500 for methods, systems, and computer program compositions that generate a plan for cannabinoid treatment operations. It is appreciated that a data engine stored in a memory device (e.g., transitory or non-transitory memory) may cause a computer processor to execute the various processing stages of the workflows discussed herein. For example, the disclosed techniques may be implemented as data engine within a medical software tool such that the data engine enables, supports, or facilitates generating a care plan for cannabinoid treatment operations based on the processes outlined herein.

At block 502, the data engine generates, one or more models that include at least one of a: a care plan parameter outlining at least a process for preparing or delivering a cannabinoid or THC composition to a biological system; health history parameter indicating health history data of the biological system; lab data parameter indicating test data of one or more laboratory tests on a biological sample of the biological system; a physician input parameter indicating contextual health analysis information of a care giver of the biological system; physiological measurement parameter derived from sensors or medical devices coupled to the biological system and which measure or analyze vitals of the biological system; and a response parameter (e.g., cannabinoid response parameter) indicating effects of one or more cannabinoid compositions on the biological system.

Turning to block 504, the data engine electronically links the one or more models to a scale comprising a plurality of therapeutic indices, the scale having a shape of a Normal distribution with a midpoint value of 500. At block 506, the data engine receives one or more of: a first input associated with the at least one parameter via a generated digital input form, the first input comprising a static input entered into one or more fields of the digital input form; and/or a second input associated with the at least one parameter, the second input comprising a dynamic input automatically received from a medical device or a medical database associated with the biological system or a caregiver of the biological system.

At block 508, the data engine determines, based on the first input or the second input, a first therapeutic index comprised in the plurality of therapeutic indices following which the data engine generates, at block 510, using the first therapeutic index, a plan (e.g., care plan) for the biological system. The plan, for example, can comprise a document or a file indicating at least one of: a procedure for titrating or preparing one or more composition doses for the biological system and/or one or more cannabinoid or THC compositions associated with the one or more cannabinoid or THC doses; delivery methods of said cannabinoid or THC compositions; guidelines on expected biological system symptoms in response to the biological system taking said one or more cannabinoid or THC compositions; safety instructions for the biological system or user taking the one or more cannabinoid or THC composition; and/or a recommendation of a combination of two or more cannabinoid or THC compositions associated with the one or more cannabinoid or THC doses.

These and other implementations may each optionally include one or more of the following features.

### Second Exemplary Workflow

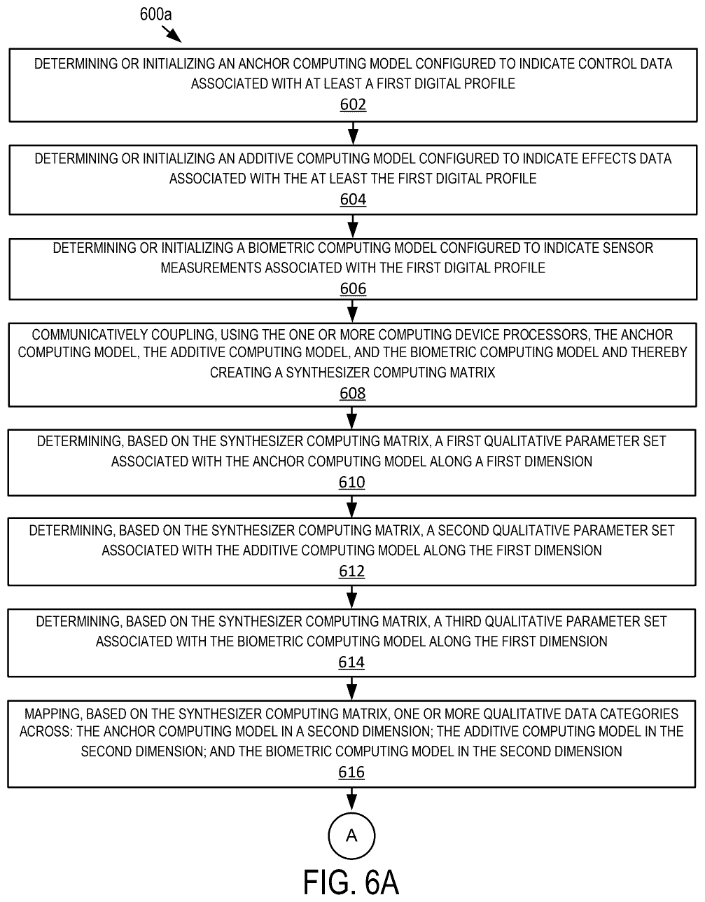

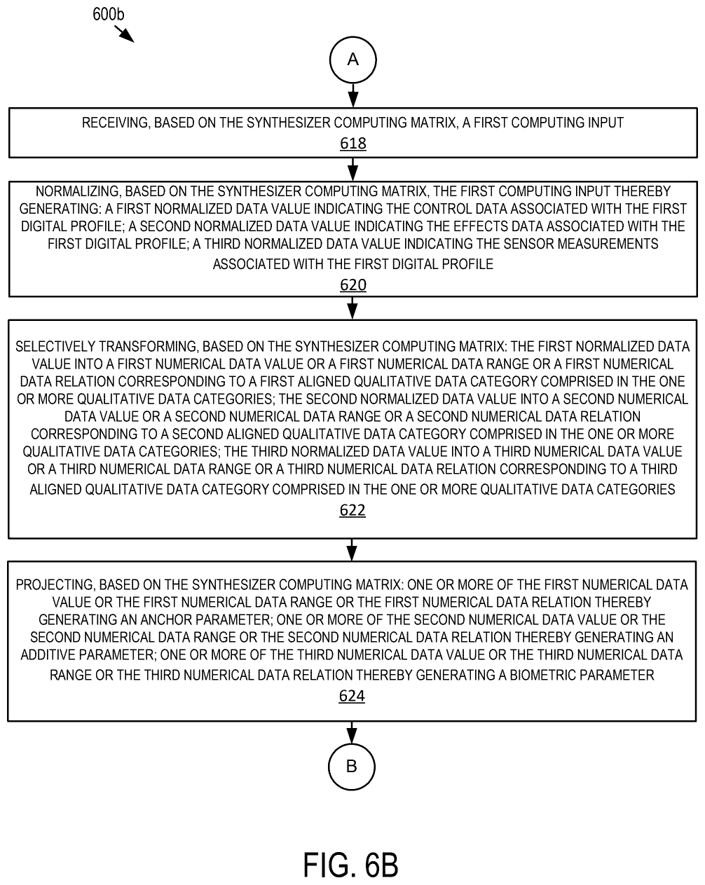

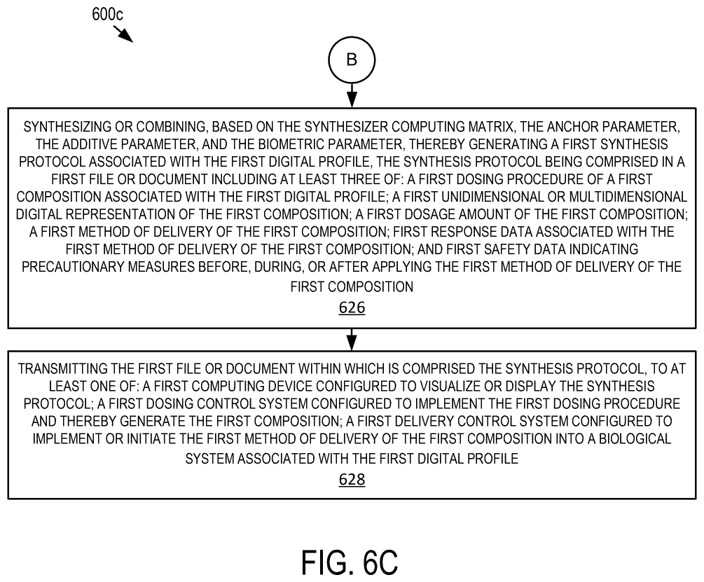

FIGS. 6A, 6B, and 6C provide exemplary workflows 600a-600c for methods, systems, and computer program products for generating a synthesis protocol associated with a digital profile. It is appreciated that a data engine stored in a memory device (e.g., transitory or non-transitory memory) may cause a computer processor to execute the various processing stages of the workflows of FIGS. 6A-6C. For example, the disclosed techniques may be implemented as data engine within a composition assessment tool such that the data engine enables, supports, or facilitates generating a synthesis protocol based on the processes outlined in FIGS. 6A-6C.

At block 602, the data engine determines or initializes an anchor computing model configured to indicate control data associated with at least a first digital profile. At block 604, the data engine determines or initializes an additive computing model configured to indicate effects data associated with the at least the first digital profile. Similarly, the data engine determines or initializes a biometric computing model configured to indicate sensor measurements associated with the first digital profile as shown at block 606.

Turning to block 608, the data engine communicatively couples or links or logically connects the anchor computing model, the additive computing model, and the biometric computing model and thereby creating a synthesizer computing matrix.

At block 610, the data engine determines, based on the synthesizer computing matrix, a first qualitative parameter set associated with the anchor computing model along a first dimension. In addition, the data engine determines, based on the synthesizer computing matrix, a second qualitative parameter set associated with the additive computing model along the first dimension as indicated at block 612. At block 614, the data engine determines, based on the synthesizer computing matrix, a third qualitative parameter set associated with the biometric computing model along the first dimension.

According to one embodiment, the data engine maps at block 616, based on the synthesizer computing matrix, one or more qualitative data categories across: the anchor computing model in a second dimension; the additive computing model in the second dimension; and the biometric computing model in the second dimension. Following this, the data engine receives (e.g., receives at a first time), at block 618, based on the synthesizer computing matrix, a first computing input. This data input may be received via a graphical user interface configured to format and transmit the first data for further processing based on the workflows of FIGS. 6A-6C.

It is appreciated that the data engine can normalize, at block 620, based on the synthesizer computing matrix, the first computing input thereby generating: a first normalized data value indicating the control data associated with the first digital profile; a second normalized data value indicating the effects data associated with the first digital profile; and a third normalized data value indicating the sensor measurements associated with the first digital profile.

At block 622, the data engine selectively transforms, based on the synthesizer computing matrix: the first normalized data value into a first numerical data value or a first numerical data range or a first numerical data relation corresponding to a first aligned or mapped qualitative data category comprised in the one or more qualitative data categories; the second normalized data value into a second numerical data value or a second numerical data range or a second numerical data relation corresponding to a second aligned or mapped qualitative data category comprised in the one or more qualitative data categories; the third normalized data value into a third numerical data value or a third numerical data range or a third numerical data relation corresponding to a third aligned or mapped qualitative data category comprised in the one or more qualitative data categories.

Turning to block 624, the data engine projects, based on the synthesizer computing matrix: one or more of the first numerical data value or the first numerical data range or the first numerical data relation thereby generating an anchor parameter; one or more of the second numerical data value or the second numerical data range or the second numerical data relation thereby generating an additive parameter; and one or more of the third numerical data value or the third numerical data range or the third numerical data relation thereby generating a biometric parameter.

At block 626, the data engine synthesizes or combines, using the one or more computing device processors and based on the synthesizer computing matrix, the anchor parameter, the additive parameter, and the biometric parameter, thereby generating a first synthesis protocol associated with the first digital profile, the synthesis protocol being comprised in a first file or document including at least three of: a first dosing procedure of a first composition associated with the first digital profile; a first unidimensional or multidimensional digital representation of the first composition; a first dosage amount of the first composition; a first method of delivery of the first composition; first response data associated with the first method of delivery of the first composition; and first safety data indicating precautionary measures before, during, or after applying the first method of delivery of the first composition.

At block 628, the data engine transmits or initiates transmission (e.g., in the case of a remote server), the first file or document within which is comprised the synthesis protocol, to at least one of: a first computing device configured to visualize or display the synthesis protocol; a first dosing control system configured to implement the first dosing procedure and thereby generate the first composition; a first delivery control system configured to implement or initiate the first method of delivery of the first composition into a biological system associated with the first digital profile. In some cases, the first file or document is locally generated on the first computing device prior to being transmitted to the first dosing control system or the first delivery control system.

In other embodiments, a system and a computer program can include or execute the method described above. These and other implementations may each optionally include one or more of the following features.

The first qualitative parameter set associated with the anchor computing model comprises one or more of: a symptom control property configured to characterize biological symptoms from known or unknown causes associated with the first digital profile; a psychosomatic property configured to characterize psychological or emotional data associated with the first digital profile; and a somatic property configured to characterize physiological data associated with the first digital profile.

It is appreciated that the second qualitative parameter set associated with the additive computing model comprises one or more of: euphoria response data associated with the first composition or a second composition relative to the first digital profile; drowsiness data associated with the first composition or the second composition relative to the first digital profile; and appetite stimulation data associated with the first composition or the second composition relative to the first digital profile.

In some embodiments, the third qualitative parameter set associated with the biometric computing model comprises one or more of: heart rate data associated with the first digital profile; and blood pressure data associated with the first digital profile.

Furthermore, the first computing input comprises one or more of: identifier data associated with the first digital profile; demographic data associated with the first digital profile; response data associated with the first digital profile; wellness data associated with the first digital profile; symptoms data associated with the first digital profile; treatment data associated with the first digital profile; medication data associated with the first digital profile; health history data associated with the first digital profile; procedure data associated with the first digital profile; sleep data associated with first digital profile; and composition use data associated with the first digital profile.

It is appreciated that the above method can further comprise generating a second synthesis protocol associated with the first digital profile or a second digital profile based on the first computing input or a second computing input (e.g., received at a second time different from the first time), wherein: the second synthesis protocol is comprised in a second file or document including at least one of: a second dosing procedure of the first composition associated with the first digital profile or a second composition associated with the first digital profile or the second digital profile; a second unidimensional or multidimensional digital representation of the first composition or a second composition; a second dosage amount of the first composition or the second composition; a second method of delivery of the first composition or the second composition; second response data associated with the second method of delivery of the first composition or the second composition; and second safety data indicating precautionary measures before, during, or after applying the second method of delivery of the first composition or the second composition.

It is further appreciated that the above method further comprises transmitting the second file or document within which is comprised the second synthesis protocol, to at least one of: the first computing device configured to visualize or display the second synthesis protocol or a second computing device configured to visualize the second synthesis protocol; the first dosing control system or a second dosing control system configured to implement the first dosing procedure and thereby generate the second composition; and the first delivery control system or a second delivery control system configured to implement or initiate the second method of delivery of the second composition into a biological system associated with the first digital profile or the second digital profile.

In some embodiments, the first synthesis protocol or the second synthesis protocol comprises a customized cannabinoid treatment protocol for one or more of the first digital profile or the second digital profile based on the first computing input or the second computing input.

Additionally, the first composition or the second composition can comprise a Tetrahydrocannabinol (THC) composition.

Moreover, the above method can further comprise predicting, based on the first synthesis protocol or the second synthesis protocol, a third synthesis protocol associated with a third computing input linked to the first digital profile or the second digital profile or a third digital profile, such that the predicting is based on a machine learning engine trained using: the synthesizer computing matrix; the first synthesis protocol; and the second synthesis protocol.

According to one embodiment, the above method further comprises: mapping, based on the synthesizer computing matrix, first synthesized data derived from the synthesizing or combining to a first index comprised in an index set; and generating, using the one or more computing device processors and based on the first index comprised in the index set, the first synthesis protocol.

It is appreciated that the index set comprises a multi-dimensional scale including one or more of: quantitative data values linked to a plurality of synthesized data including the first synthesized data or second synthesized data that is generated based on a second computing input that is different from the first computing input; and qualitative data values characterizing range information for each data value comprised in the quantitative data values linked to the plurality of synthesized data.

It is further appreciated that the range information comprises: a first value range indicating low composition effects data; a second value range indicating medium composition effects data; a third value range indicating optimal composition effects data; a fourth value range indicating high composition effects data; and a fifth value range indicating severe composition effects data.

In some cases, the index set is statistically Normally distributed or comprises a scaled Normal distribution.

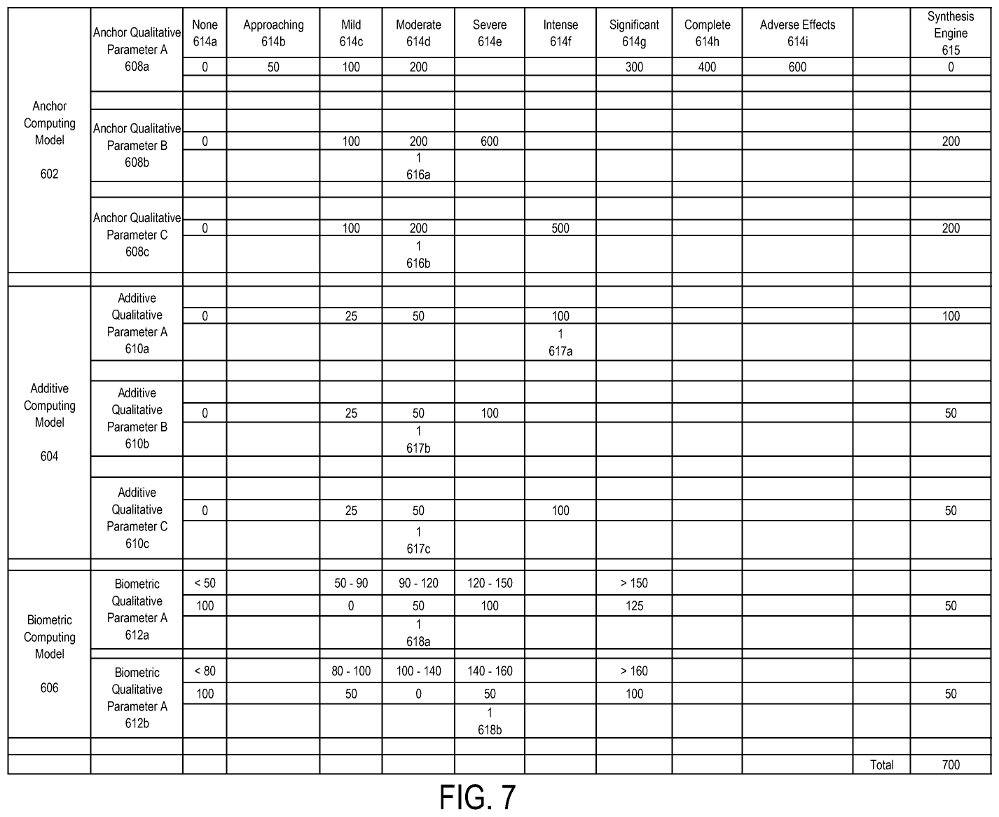

FIG. 7 provides a rendition of data elements associated with the disclosed synthesizer computing matrix according to some embodiments. As can be seen in this figure, the synthesizer computing matrix can comprise a plurality of computing models 702. 704, 706, etc., in a first dimension. In the illustrated embodiment, the plurality of computing models 702, 704, 706, etc., are organized or otherwise aligned or structured in a first dimension (e.g., a y-axis dimension). It is appreciated that the plurality of computing models can have a corresponding plurality of qualitative parameter sets 708a . . . 708c, 710a . . . 710c, and 712a . . . 712b. It is further appreciated that the plurality of computing models 702, 704, 706, etc., can be mapped to a plurality of qualitative data categories 714a . . . 714i. The plurality of qualitative data categories 714a . . . 714i can comprise varying levels of degree parameters that define or otherwise characterize an intensity level associated with the plurality of computing models 702, 704, 706, etc.

When an input is received, the input may be normalized to generate one or more normalized data values 716a, 716b, 717a, 717b, 718a, 718b, etc. As seen in the illustrated example, the normalized value for each of the normalized data values 716a, 716b, 717a, 717b, 718a, 718b is 1. However, the normalized values can be adapted or otherwise dynamically varied, changed, or configured to have data values that enable differentiating a plurality of data inputs and/or tying more closely, specific data inputs to specific computing models associated with the disclosed synthesizer computing matrix.

Also shown in this figure is a synthesis engine 715 which can include logic executable by a computer processor to transform the normalized data values of the synthesizer computing matrix into numerical data as well as project said numerical data to generate one or more parameters associated with the various computing models of the synthesizer computing matrix. For example, the parameters can comprise an aggregate of numerical data generated from the transformation of the normalized data values for each of the various computing models associated with, or comprised in the disclosed synthesizer computing matrix. These values can be further synthesized or combined to generate synthesized data which are tied to, for example, an index set associated with, for example, the index set discussed in association with FIG. 4.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to explain the principles of the disclosed subject-matter and its practical applications, to thereby enable others skilled in the art to use the technology disclosed and various embodiments with various modifications as are suited to the particular use contemplated.

It is appreciated that the term optimize/optimal and its variants (e.g., efficient or optimally) may simply indicate improving, rather than the ultimate form of ‘perfection’ or the like.

### Additional Embodiments

The disclosed methods and systems beneficially facilitate assessing, quantifying, and recommending data indicating safe compositions (e.g., cannabinoids, terpenes, flavonoids, etc.) dose and/or ingredients for said compositions, amount of said compositions, timing of delivery of said compositions to biological systems, consumption or delivery method of said compositions, extraction methods for said compositions, and safely discharging or delivering said compositions to achieve maximum effect with minimal adverse effects. In particular, the generated synthesis protocol discussed above can include the foregoing recommended data which can be further used for research, data insights, consumer research, product research, biological system safety, as well as exploration of benefits, therapeutic effects risks, and side effects for both long and short terms of composition use. Additionally, the disclosed methods and systems are similarly applicable to recommending the aforementioned data for other plant-based or non-plant-based supplements like nutraceuticals and functional supplements like functional mushrooms.

Additionally, the disclosed methods and systems can be beneficially used in a variety of diverse scenarios. For example, the disclosed methods and systems can be leveraged using digital profiles of patients consumers, and/or patients with or without caregivers via a web application or phone application. Digital profiles of clinicians and healthcare workers can also be used to access and implement the disclosed methods and systems in conjunction with digital profiles of patients or users to develop synthesis protocols associated with biological systems of the patients or users in inpatient acute care situations, inpatient intensive care situations, outpatient clinic settings, nursing facilities, long-term acute care facilities, research studies, home care, hospice care, palliative care, and/or through a telehealth platform/botanical care plans. In some embodiments, the disclosed methods and systems can be used on computing systems including: complex or noncomplex telehealth platforms, smart watches and smart tools, medical devices, wellness applications, health and wellness databases, supplement brand and retail websites, a web application or phone application for patients and caregivers for personal or home use, other applications/plug-ins, 3rd party applications and website, as well as other electronic medical records.

The disclosed methods and systems beneficially enable generating synthesis protocols for compositions that include cannabinoids, terpenes, hemp, and titrating compositions with or without THC, as well as other botanical and plant-based supplements like nutraceuticals and functional mushrooms for symptom relief. In particular, the disclosed methods and systems provide a structured approach to addressing underlying chemical imbalances (e.g., endocannabinoid imbalances) and finding accurate composition dosing, route of administration, composition and terpene combinations within an index set (e.g., therapeutic index set) to provide symptom relief for chronic pain, neuropathic pain, anxiety, spasticity, insomnia, depression, headaches, nausea, appetite stimulation, ADHD, and stress, MS, Parkinson Disease, PTSD, blood pressure reduction, migraines, glaucoma, irritable bowel disease, Inflammatory bowel disease, nausea, vomiting, appetite stimulation, menopause symptom management, endometriosis symptoms, male and female sexual health. impact-social anxiety (obviously related to anxiety mentioned), seizures, and autism spectrum disorder behaviors while also minimizing acute side effects and identifying other insights like the cause of Cannabis Hyperemesis Syndrome and how to prevent or treat it.

According to one embodiment, inputs (e.g., first inputs, second inputs, third inputs, etc.) may be received using the disclosed systems. Said inputs can include subjective inputs and/or objective inputs at designated intervals (e.g., inputs received at two or more different times) which can be used to generate synthesis data (e.g., a data value associated with a composition, a data property associated with the disclosed composition, a data quantifier for predicting property values of the disclosed composition, a data qualifier for characterizing property values of the disclosed compositions relative to one or more digital profiles) which can be used for recommending and/or providing instructions on optimally dosing biological systems associated with the one or more digital profiles.

According to one embodiment, the disclosed methods and systems can receive and/or analyze one or more of the following computing data inputs prior to generating the synthesis protocol:
Identifier data associated with a digital profile.
Age data associated with the digital profile.
Allergy data associated with the digital profile.
Health and Wellness data associated with the digital profile.
Symptoms or discomfort data associated with the digital profile.
Symptom management success data associated with the digital profile.
Symptom management failure data associated with the digital profile.
Medical history data associated with the digital profile.
Hospitalization data associated with the digital profile.
Rationale data associated with said hospitalization data.
Prior surgical history data associated with the digital profile.
Future surgical history data associated with the digital profile.
Family medical history data associated with the digital profile.
Schizophrenic history data, heart disease history data, diabetes history data, or cardiac history data associated with the digital profile.
Height and weight data associated with the digital profile.
Medication data associated with the digital profile.
Side effect data associated with said medication data.
Vitamin or supplement intake data associated with the digital profile.
Side effect data associated with said vitamin or supplement intake data.
Holistic treatment data associated with the digital profile.
Effect data associated with said holistic treatment data.
Diet data associated with the digital profile.
Exercise data with attendant method of exercise data associated with the digital profile.
Personality data associated with the digital profile.
Sleep data associated with the digital profile.
Rest data associated with the sleep data.
Cannabis or CBD use history data associated with the digital profile.
Method of delivery data associated with said cannabis or CBD use history data.
Quantity data associated with said cannabis or CBD use history data.
Effects data associated with said cannabis or CBD use history data.
Cannabinoid use history data associated with the digital profile.
Cannabis composition data associated with the digital profile.
Cannabis or CBD composition preference data associated with the digital profile.
Aversion data associated with the cannabis or CBD composition preference data.
Medical Cannabis Card data associated with the digital profile.

According to one embodiment, the disclosed methods and systems can be used to refine or optimize generation or updating of the synthesis data based on one or more of:
Specialized Dementia Assessment and Screening data.
GAD-7 Score data.
PSQI—Pittsbug Sleep Quality Index.
Anxiety Symptom Assessment data.
Pain Scale data.
Neurologic Assessment Intake data.
Estrogen Imbalance—Hormone Health Intake data.
Testosterone Imbalance Intake Patient Assessment Form data.
Pain Score data.
Patient Follow-Up Assessment data.

According to one embodiment, the disclosed methods include process where previous synthesis data associated with a digital profile is used to inform future synthesis protocol generation for the digital profile. For example, the prior synthesis data may me leveraged to make recommendations included in a synthesis protocol in combination with a literature review of existing research, reviews of previous responses from similar or dissimilar digital profiles, nursing therapeutic interventions, other endocannabinoid stimulating plant based interventions, safety data, drug-drug interactions data, and other variables to generate the updated synthesis protocol.

According to one embodiment, the disclosed synthesis protocol may be stored in a in the pharmaceutical data storage bin associated with the digital profile.

The disclosed methods and systems find application in Home Health Care, Home Hospice, Doctors' and Dentists' offices, Medical Spas, Surgical Centers, Day spas, Urgent Care, Assisted Living, Memory Care, Substance Abuse Rehab Centers School nurses offices (e.g., medical cannabis is legal in most states), Senior Day Programs, and other types of unaccounted for outpatient centers, etc.

According to one embodiment, the disclosed methods and systems involve using, based on synthesis data, an index set having a plurality of quantitative values. For example, the plurality of quantitative values can range from 1 to 1000 and can be split into a plurality (e.g., at least 3, or 4, or 5) distinct index sets or zones representing varying degrees or levels of therapeutic effect from subtherapeutic to supratherapeutic delivery of compositions. According to one embodiment, an optimal zone comprised in the distinct index sets with be comprised in a midrange (e.g., between 420-580) can be associated with a plurality of digital profiles and can indicate an optimal response for delivery compositions based on one or more synthesis protocols to biological systems associated with the plurality of digital profiles. In one embodiment, the index set is based on both objective markers such as vital signs and subjective observations regarding a set of physiological symptoms and potential side effects such as level of drowsiness or anxiety, each measured using a 7-point Likert scale associated with one or more of the plurality of digital profiles. According to one embodiment, the index set can have a first axis in a first dimension that indicates one or more quantitative values corresponding to a dosing parameter in a second dimension indicating side effect data and optimal therapeutic effect data.

In one embodiment, data inputs associated with a digital profile are captured, and synthesis data is determined at multiple data points or times: before a biological system of the digital profile consumes a composition to get baseline data and then again right after consuming the composition to measure the composition's effect. Additional synthesis data may be captured at time intervals determined by the specific route of administration of the composition to generate a complete picture of the impact over the complete duration of action for the composition in question. The original and additional synthesis data may be combined to produce a final synthesis data using a weighting strategy informed by how long it has been since the biological system's last intake of the composition. If the final synthesis data falls below, a given threshold (e.g., 150 on a scale associated with the index set), a new dose would be considered subtherapeutic. In contrast, a final synthesis data above 750 threshold would indicate that the dose was supratherapeutic and likely that the biological system experienced significant adverse effects. The goal is to achieve synthesis data corresponding to a range between 150 and 750 with an optimal range between 420 and 580. Based on this final synthesis data, adjustments to the dose, route, or other product attributes can be made to achieve an optimal result the next time. By capturing and aggregating the data attributes associated with one or more digital profiles, composition attributes, session feedback, and synthesis data, machine learning can be leveraged to improve the precision and accuracy of the synthesis data and predict a given biological systems response to a composition (e.g., cannabis composition) with attendant dosage data.

According to one embodiment, the disclosed methods of systems provide a total or final synthesis data that can be statistically weighted based on multiple inputs. For example, a biometric computing model associated with the disclosed synthesizer computing matric can be configured to have a greater statistical weights or parameters or scaled values than data values associated with the synthesizer computing matrix. In some embodiments, the disclosed methods can involve recommending adjustments to synthesis protocols.

Furthermore, the following classifications can be applied to the various zones or index ranges shown in FIG. 4.
Zone 0-250
At most 2 criteria points
None or few noticeable sensory or somatic sensations
No psychoactive effects
Drowsiness Relaxation
Exemplary corresponding Likert Score: 1-3
Zone 250-420:
At least 1 criteria point
Mild psychoactive side effects
Mild reported or observed euphoria
Mild sensory and/or somatic sensations
Exemplary corresponding Likert Score: 2-4
Zone 420-580
1 major criteria point and no more than 2 minor criteria points
Major:
Expected exemplary Likert Score: 4-6
Reported euphoria
Relaxation
Appetite stimulation
Minor:
Time dilation
Heart Rate 90-110 BPM
No anxiety or mild anxiety
Drowsiness Decreased systolic BP
Mild to moderate Somatic Sensations
Zone 580-750:
At least 1 criteria point
Moderate to severe psychoactive side effects
Moderate increase in anxiety
Moderately intense sensory and/or somatic sensations Reported Likert Score: 3-6
Zone 750-1000:
At least 2 or more criteria point:
Severe Anxiety
Hallucinations
Tremors
Heart Rate>120 BPM
Systolic BP>140 or <90
Uncomfortable somatic sensations Nausea and/or Vomiting
Exemplary corresponding Likert Score: 0-5

According to one embodiment, the disclosed synthesis protocol is derived from, or includes an index set ranging from 0-1000 with attendant zones (Grey, Blue, Green, Yellow, or Red) which are further explained below with exemplary recommendations:
The Grey Zone: 0-250
Outputs: Increase THC by 50%,
Add Terpenes,
Increase other Cannabinoids by 50% (CBD, CBG),
Add a 20:1 ratio (and other interventions like Avoid caffeine and grapefruit with the supplement, take with a meal used to enhance efficacy)
Blue Zone: 250-420
Outputs: Increase THC by 50%,
Add Terpenes,
Increase other Cannabinoids by 50% (CBD, CBG), Add a 20:1 ratio (And other interventions like Avoid caffeine and grapefruit with supplement use to enhance efficacy).
Green Zone: 420-580:
Stay at this dose, product, route.
Yellow Zone: 580-750
Outputs: Decrease THC by 25%,
Add Terpenes,
Increase other Cannabinoids by 25%, 50% (CBD, CBG), Decrease other cannabinoids by 25% or 50% Add a 20:1 ratio (And other interventions like Separate other supplements and meds by 30 minutes or add the supplement PEA).
Red Zone>750
Decrease THC by 50%,
Add Terpenes,
Increase other Cannabinoids by 10% (CBD, CBG), (And other interventions like: Separate other supplements and meds by 30 minutes or add the supplement PEA)

It is appreciated that the disclosed methods and systems provide therapeutic effects of botanicals, cannabis, and hemp by capture and/or determining real-time assessments of what happened to the biological systems while saving users time, money, and avoidance of continued use of incorrect or unsafe cannabinoid compositions. A user may be able to obtain knowledge of ideal compositions, route, and dosage of cannabinoids, ingredients, terpenes, and flavonoids based on the disclosed synthesis protocol. The disclosed methods and systems can quantify composition titration data for clinicians, caregivers, and other users. Having synthesis data that corresponds to or otherwise quantifies composition data relative to a given biological system of a digital profile provides a compelling tool for assessment, drug development, research, and data analytics that clearly notate the level of efficacy or lack of efficacy of a cannabinoid composition, for example. Additionally, the disclosed methods and systems provide tools that can be used by the individual users, caregivers, and medical professionals to avoid side effects and risks associated with delivering cannabinoid compositions to biological systems. The disclosed synthesis protocol can also provide optimal dosing times for delivering cannabinoid compositions together with various physiological effects data of different cannabinoid compositions, ingredients, and supplements customized for a digital profile.

According to one embodiment, the synthesis data and/or digital profile data may be securely stored on a local computing platform associated with a plurality of users or a cloud computing platform associated with the plurality of users. In some cases, prior to using the synthesis protocol for additional research or optimization of delivery of cannabinoid compositions, the synthesis protocol is de-identified of personal health information of corresponding digital profiles. In one embodiment, no de-identification of the personal health information is conducted prior to using the synthesis protocol to optimize cannabinoid composition delivery research.

Furthermore, the functions or operations described above can be implemented in digital electronic circuitry, in computer software, firmware or hardware. In particular, the disclosed techniques can be implemented using one or more computer program compositions. The computer program compositions, in some embodiments, comprises non-transitory computer-readable media comprising code configured to execute the disclosed approach. Programmable processors and computers can be included in or packaged as mobile devices according to some embodiments. The processes and logic flows can be performed by one or more programmable processors and by one or more programmable logic circuitry. General and special purpose computing devices and storage devices can be interconnected through communication networks.

Moreover, although the terms first, second, etc., may be used herein to describe various elements, these elements should not be limited by these terms. These terms are used to distinguish one element from another. For example, a first object or step could be termed a second object or step, and, similarly, a second object or step could be termed a first object or step, without departing from the scope of the invention. The first object or step, and the second object or step, are both objects or steps, respectively, but they are not to be considered the same object or step.

The terminology used in the description herein is for the purpose of describing particular embodiments and is not intended to be limiting. As used in the description of the invention and the appended claims, the singular forms “a,” “an” and “the” are intended to include the plural forms as well, unless the context clearly indicates otherwise. It will also be understood that the term “and/or” as used herein refers to and encompasses any possible combination of one or more of the associated listed items. It will be further understood that the terms “includes,” “including,” “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context.

Those with skill in the art will appreciate that while some terms in this disclosure may refer to absolutes, e.g., all source receiver traces, each of a plurality of objects, etc., the methods and techniques disclosed herein may also be performed on fewer than all of a given thing, e.g., performed on one or more components and/or performed on one or more source receiver traces. Accordingly, in instances in the disclosure where an absolute is used, the disclosure may also be interpreted to be referring to a subset.

What is claimed is:

1. A method for generating a synthesis protocol associated with a digital profile, the method comprising:
determining or initializing, using one or more computing device processors, an anchor computing model configured to indicate control data associated with at least a first digital profile;
determining or initializing, using the one or more computing device processors, an additive computing model configured to indicate effects data associated with the at least the first digital profile;
determining or initializing, using the one or more computing device processors, a biometric computing model configured to indicate sensor measurements associated with the first digital profile;
communicatively coupling, using the one or more computing device processors, the anchor computing model, the additive computing model, and the biometric computing model and thereby creating a synthesizer computing matrix;
determining, using the one or more computing device processors and based on the synthesizer computing matrix, a first qualitative parameter set associated with the anchor computing model along a first dimension;
determining, using the one or more computing device processors and based on the synthesizer computing matrix, a second qualitative parameter set associated with the additive computing model along the first dimension;
determining, using the one or more computing device processors and based on the synthesizer computing matrix, a third qualitative parameter set associated with the biometric computing model along the first dimension;
mapping, using the one or more computing device processors and based on the synthesizer computing matrix, one or more qualitative data categories across:
the anchor computing model in a second dimension,
the additive computing model in the second dimension, and
the biometric computing model in the second dimension;
receiving, using the one or more computing device processors based on the synthesizer computing matrix, a first computing input;
normalizing, using the one or more computing device processors and based on the synthesizer computing matrix, the first computing input thereby generating:
a first normalized data value indicating the control data associated with the first digital profile,
a second normalized data value indicating the effects data associated with the first digital profile,
a third normalized data value indicating the sensor measurements associated with the first digital profile;
selectively transforming, using the one or more computing device processors and based on the synthesizer computing matrix:
the first normalized data value into a first numerical data value or a first numerical data range or a first numerical data relation corresponding to a first aligned qualitative data category comprised in the one or more qualitative data categories,
the second normalized data value into a second numerical data value or a second numerical data range or a second numerical data relation corresponding to a second aligned qualitative data category comprised in the one or more qualitative data categories,
the third normalized data value into a third numerical data value or a third numerical data range or a third numerical data relation corresponding to a third aligned qualitative data category comprised in the one or more qualitative data categories;
projecting, using the one or more computing device processors and based on the synthesizer computing matrix:
one or more of the first numerical data value or the first numerical data range or the first numerical data relation thereby generating an anchor parameter,
one or more of the second numerical data value or the second numerical data range or the second numerical data relation thereby generating an additive parameter,
one or more of the third numerical data value or the third numerical data range or the third numerical data relation thereby generating a biometric parameter,
synthesizing or combining, using the one or more computing device processors and based on the synthesizer computing matrix, the anchor parameter, the additive parameter, and the biometric parameter, thereby generating a first synthesis protocol associated with the first digital profile, the synthesis protocol being comprised in a first file or document including at least three of:
a first dosing procedure of a first composition associated with the first digital profile,
a first unidimensional or multidimensional digital representation of the first composition,
a first dosage amount of the first composition,
a first method of delivery of the first composition,
first response data associated with the first method of delivery of the first composition, and
first safety data indicating precautionary measures before, during, or after applying the first method of delivery of the first composition; and
transmitting, using the one or more computing device processors, the first file or document within which is comprised the synthesis protocol, to at least one of:
a first computing device configured to visualize or display the synthesis protocol,
a first dosing control system configured to implement the first dosing procedure and thereby generate the first composition,
a first delivery control system configured to implement or initiate the first method of delivery of the first composition into a biological system associated with the first digital profile.

2. The method of claim 1, wherein the first qualitative parameter set associated with the anchor computing model comprises one or more of:
a symptom control property configured to characterize biological symptoms from known or unknown causes associated with the first digital profile,
a psychosomatic property configured to characterize psychological or emotional data associated with the first digital profile, and
a somatic property configured to characterize physiological data associated with the first digital profile.

3. The method of claim 1, wherein the second qualitative parameter set associated with the additive computing model comprises one or more of:
euphoria response data associated with the first composition or a second composition relative to the first digital profile,
drowsiness data associated with the first composition or the second composition relative to the first digital profile, and
appetite stimulation data associated with the first composition or the second composition relative to the first digital profile.

4. The method of claim 1, wherein the third qualitative parameter set associated with the biometric computing model comprises one or more of:
heart rate data associated with the first digital profile, and
blood pressure data associated with the first digital profile.

5. The method of claim 1, wherein the first computing input comprises one or more of:
identifier data associated with the first digital profile,
demographic data associated with the first digital profile,
response data associated with the first digital profile,
wellness data associated with the first digital profile,
symptoms data associated with the first digital profile,
treatment data associated with the first digital profile,
medication data associated with the first digital profile,
health history data associated with the first digital profile,
procedure data associated with the first digital profile,
sleep data associated with first digital profile, and
composition use data associated with the first digital profile.

6. The method of claim 1, further comprising generating a second synthesis protocol associated with the first digital profile or a second digital profile based on the first computing input or a second computing input, wherein:
the second synthesis protocol is comprised in a second file or document including at least one of:
a second dosing procedure of the first composition associated with the first digital profile or a second composition associated with the first digital profile or the second digital profile,
a second unidimensional or multidimensional digital representation of the first composition or a second composition,
a second dosage amount of the first composition or the second composition,
a second method of delivery of the first composition or the second composition,
second response data associated with the second method of delivery of the first composition or the second composition, and
second safety data indicating precautionary measures before, during, or after applying the second method of delivery of the first composition or the second composition.

7. The method of claim 6, further comprising transmitting, using the one or more computing device processors, the second file or document within which is comprised the second synthesis protocol, to at least one of:
the first computing device configured to visualize or display the second synthesis protocol or a second computing device configured to visualize the second synthesis protocol,
the first dosing control system or a second dosing control system configured to implement the first dosing procedure and thereby generate the second composition, and
the first delivery control system or a second delivery control system configured to implement or initiate the second method of delivery of the second composition into a biological system associated with the first digital profile or the second digital profile.

8. The method of claim 6, wherein the first synthesis protocol or the second synthesis protocol comprises a customized cannabinoid treatment protocol for one or more of the first digital profile or the second digital profile based on the first computing input or the second computing input.

9. The method of claim 6, wherein the first composition or the second composition comprises a Tetrahydrocannabinol (THC) composition.

10. The method of claim 6, further comprising predicting, using the one or more computing device processors and based on the first synthesis protocol or the second synthesis protocol, a third synthesis protocol associated with a third computing input linked to the first digital profile or the second digital profile or a third digital profile, wherein the predicting is based on a machine learning engine trained using:
the synthesizer computing matrix,
the first synthesis protocol, and
the second synthesis protocol.

11. The method of claim 1, further comprising:
mapping, using the one or more computing device processors and based on the synthesizer computing matrix, first synthesized data derived from the synthesizing or combining to a first index comprised in an index set; and
generating, using the one or more computing device processors and based on the first index comprised in the index set, the first synthesis protocol.

12. The method of claim 11, wherein the index set comprises a multi-dimensional scale including one or more of:
quantitative data values linked to a plurality of synthesized data including the first synthesized data or second synthesized data that is generated based on a second computing input that is different from the first computing input, and
qualitative data values characterizing range information for each data value comprised in the quantitative data values linked to the plurality of synthesized data.

13. The method of claim 12, wherein the range information comprise:
a first value range indicating low composition effects data,
a second value range indicating medium composition effects data,
a third value range indicating optimal composition effects data,
a fourth value range indicating high composition effects data, and
a fifth value range indicating severe composition effects data.

14. The method of claim 11, wherein the index set is statistically Normally distributed.

15. A system for generating a synthesis protocol associated with a digital profile, the system comprising:
one or more computing system processors; and
at least one memory storing instructions associated with a synthesizer computing matrix, that when executed by the one or more computing system processors cause the one or more computing system processors to:
determine or initialize an anchor computing model configured to indicate control data associated with at least a first digital profile;
determine or initialize an additive computing model configured to indicate effects data associated with the at least the first digital profile;
determine or initialize a biometric computing model configured to indicate sensor measurements associated with the first digital profile;
communicatively couple the anchor computing model, the additive computing model, and the biometric computing model thereby creating the synthesizer computing matrix;
determine, based on the synthesizer computing matrix, a first qualitative parameter set associated with the anchor computing model along a first dimension;
determine, based on the synthesizer computing matrix, a second qualitative parameter set associated with the additive computing model along the first dimension;
determine, based on the synthesizer computing matrix, a third qualitative parameter set associated with the biometric computing model along the first dimension;
map, based on the synthesizer computing matrix, one or more qualitative data categories across:
the anchor computing model in a second dimension,
the additive computing model in the second dimension, and
the biometric computing model in the second dimension;
receive, based on the synthesizer computing matrix, a first computing input;
normalize, based on the synthesizer computing matrix, the first computing input thereby generating:
a first normalized data value indicating the control data associated with the first digital profile,
a second normalized data value indicating the effects data associated with the first digital profile,
a third normalized data value indicating the sensor measurements associated with the first digital profile;
selectively transform, based on the synthesizer computing matrix:
the first normalized data value into a first numerical data value or a first numerical data range or a first numerical data relation corresponding to a first aligned qualitative data category comprised in the one or more qualitative data categories,
the second normalized data value into a second numerical data value or a second numerical data range or a second numerical data relation corresponding to a second aligned qualitative data category comprised in the one or more qualitative data categories,
the third normalized data value into a third numerical data value or a third numerical data range or a third numerical data relation corresponding to a third aligned qualitative data category comprised in the one or more qualitative data categories;
project, based on the synthesizer computing matrix:
one or more of the first numerical data value or the first numerical data range or the first numerical data relation thereby generating an anchor parameter,
one or more of the second numerical data value or the second numerical data range or the second numerical data relation thereby generating an additive parameter,
one or more of the third numerical data value or the third numerical data range or the third numerical data relation thereby generating a biometric parameter,
synthesize or combine, based on the synthesizer computing matrix, the anchor parameter, the additive parameter, and the biometric parameter thereby generating a first synthesis protocol associated with the first digital profile, the synthesis protocol being comprised in a first file or document comprising at least one of:
a first dosing procedure of a first composition associated with the first digital profile,
a first unidimensional or multidimensional digital representation of the first composition,
a first dosage amount of the first composition,
a first method of delivery of the first composition,
first response data associated with the first method of delivery of the first composition, and
first safety data indicating precautionary measures before, during, or after applying the first method of delivery of the first composition; and
transmit the first file or document within which is comprised the synthesis protocol, to at least one of:
a first computing device configured to visualize or display the synthesis protocol,
a first dosing control system configured to implement the first dosing procedure and thereby generate the first composition,
a first delivery control system configured to implement or initiate the first method of delivery of the first composition into a biological system associated with the first digital profile.

16. The system of claim 15, wherein the first computing input comprises one or more of:
identifier data associated with the first digital profile,
demographic data associated with the first digital profile,
response data associated with the first digital profile,
wellness data associated with the first digital profile,
symptoms data associated with the first digital profile,
treatment data associated with the first digital profile,
medication data associated with the first digital profile,
health history data associated with the first digital profile,
procedure data associated with the first digital profile,
sleep data associated with first digital profile, and
composition use data associated with the first digital profile.

17. The system of claim 15, wherein the first synthesis protocol comprises a customized cannabinoid treatment protocol for the first digital profile based on the first computing input.

18. The system of claim 15, wherein the instructions associated with the synthesizer computing matrix are executed by the one or more computing system processors to:
map, based on the synthesizer computing matrix, first synthesized data derived from the synthesizing or combining to a first index comprised in an index set; and
generate, based on the first index comprised in the index set, the first synthesis protocol.

19. The system of claim 18, wherein the index set comprises a multi-dimensional scale including one or more of:
quantitative data values linked to a plurality of synthesized data including the first synthesized data or second synthesized data that is generated based on a second computing input that is different from the first computing input, and
qualitative data values characterizing range information for each data value comprised in the quantitative data values linked to the plurality of synthesized data.

20. The system of claim 19, wherein the range information comprise:
a first value range indicating low composition effects data,
a second value range indicating medium composition effects data,
a third value range indicating optimal composition effects data,
a fourth value range indicating high composition effects data, and
a fifth value range indicating severe composition effects data.
